(19) United States
(12) Patent Application Publication
(10) Pub. No.: US 2024/0166746 A1
(43) Pub. Date: May 23, 2024

(54) DOSAGE REGIMEN OF MAdCAM-1 ANTIBODY FOR TREATMENT OF NON-ALCOHOLIC STEATOHEPATITIS

(71) Applicant: Takeda Pharmaceutical Company Limited, Osaka-Shi (JP)

(72) Inventors: Peter Nagy, Zug (CH); Zoltan Derdak, Attleboro, MA (US)

(21) Appl. No.: 18/515,766

(22) Filed: Nov. 21, 2023

Related U.S. Application Data
(60) Provisional application No. 63/597,487, filed on Nov. 9, 2023.
(60) Provisional application No. 63/502,322, filed on May 15, 2023.
(60) Provisional application No. 63/384,723, filed on Nov. 22, 2022.

(51) Int. Cl. C07K 16/28; A61K 39/00; A61P 1/16; G01N 33/50

(52) U.S. Cl. CPC C07K 16/2803; A61P 1/16; G01N 33/5008; A61K 2039/505; A61K 2039/545; C07K 2317/565

(57) ABSTRACT

The present disclosure provides, among other things, treatment regimens of MAdCAM-1 antibodies for patients susceptible to or diagnosed with non-alcoholic steatohepatitis.

### CROSS REFERENCE TO RELATED APPLICATIONS

This application claims priority to, and the benefit of U.S. provisional application No. 63/384,723, filed on Nov. 22, 2022, U.S. provisional application No. 63/502,322, filed on May 15, 2023, and U.S. provisional application No. 63/597,487, filed on Nov. 9, 2023, the contents of each of which is hereby incorporated by reference in its entirety.

### INCORPORATION-BY-REFERENCE OF SEQUENCE LISTING

The content of the text file name “SHR-2026_Sequence Listing.xml”, which was created on Nov. 20, 2023, and is 56 KB in size, is hereby incorporated-by-reference in its entirety.

### TECHNICAL FIELD

The present invention relates to dosage regimens of MAdCAM-1 antibodies for the treatment of non-alcoholic steatohepatitis (NASH).

### BACKGROUND

Non-alcoholic steatohepatitis is a non-benign disorder characterized by substantial health risks. Subjects diagnosed with NASH are at significantly increased risk of morbidity and mortality. More specifically, NASH is characterized by increased risk of cardiovascular and liver-related mortality. NASH can lead to cirrhosis, which in turn can result in fluid retention, muscle wasting, bleeding from the intestines, and liver failure. Liver transplantation is the only treatment for advanced cirrhosis with liver failure, with NASH is currently the number two reason for liver transplants.

Accordingly, there is a continuing need for therapeutic agents for the treatment of NASH.

### SUMMARY OF THE INVENTION

The present invention relates to dosage regimen for the treatment of a patient susceptible to or diagnosed with non-alcoholic steatohepatitis (NASH) comprising administering to the patient a therapeutic dose of a MAdCAM-1 antibody.

In one aspect, the present invention provides a method for the treatment of a patient susceptible to or diagnosed with NASH comprising administering to the patient a therapeutic dose of between about 20 mg and about 125 mg of the MAdCAM-1 antibody. In some embodiments, the method comprises administering a subsequent dose of a MAdCAM-1 antibody.

In some embodiments, either or both of the therapeutic dose and the subsequent dose of the MAdCAM-1 antibody is dosed at a range whose lower limit is 1 mg, 2 mg, 2.25 mg, 5 mg, 6 mg, 7 mg, 7.5 mg, 8 mg, 9 mg, 10 mg, 12 mg, 15 mg, 20 mg, 22.5 mg, 25 mg, 30 mg, 35 mg, 45 mg, 50 mg, 55 mg, 65 mg, 65 mg, 70 mg, 75 mg, 80 mg, 85 mg, 90 mg, 95 mg, 100 mg, 125 mg, 150 mg, 175 mg, 200 mg, or 225 mg and whose upper limit is 22.5 mg, 25 mg, 30 mg, 35 mg, 40 mg, 45 mg, 50 mg, 55 mg, 60 mg, 65 mg, 70 mg, less than 75 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, or 750 mg.

In some embodiments, the method includes administering to the patient the therapeutic dose of 22.5 mg or 75 mg of MAdCAM-1 antibody. In some embodiments, the method includes administering to the patient the therapeutic dose of 75 mg of MAdCAM-1 antibody.

In some embodiments, the MAdCAM-1 antibody comprises a light chain CDR1 of SEQ ID NO: 11; a light chain CDR2 of SEQ ID NO: 12; and a light chain CDR3 of SEQ ID NO: 13; and a heavy chain CDR1 of SEQ ID NO: 14; a heavy chain CDR2 of SEQ ID NO: 15; and a heavy chain CDR3 of SEQ ID NO: 16.

In some embodiments, the MAdCAM-1 antibody comprises a variable light chain of SEQ ID NO: 3, and a variable heavy chain of SEQ ID NO: 4.

In some embodiments, the MAdCAM-1 antibody comprises a light chain SEQ ID NO: 1 and a heavy chain SEQ ID NO: 2.

In some embodiments, the subsequent dose is administered at an amount that is same or less than the therapeutic dose, and the subsequent dose is administered between about 1 week and about 12 weeks after the therapeutic dose. In some embodiments, the subsequent dose is administered every 4 weeks. In some embodiments, the subsequent dose is administered every 8 weeks.

In some embodiments, the MAdCAM-1 antibody is administered to the patient subcutaneously. In some embodiments, the MAdCAM-1 antibody is administered to the patient intravenously.

In some embodiments, the patient is not taking a TNF antagonist or TNF inhibitor.

In one aspect, the MAdCAM-1 antibody is for use in the methods described herein.

In one aspect, a pharmaceutical composition comprises the MAdCAM-1 antibody described herein.

In one aspect, use of the MAdCAM-1 antibody is in the manufacturing of a medicament for the treatment of non-alcoholic steatohepatitis.

In another aspect, the present invention provides a method for the treatment of a patient susceptible to or diagnosed with non-alcoholic steatohepatitis (NASH) comprising administering to the patient a therapeutic dose of 22.5 mg or 75 mg of a MAdCAM-1 antibody comprising a light chain CDR1 of SEQ ID NO: 11; a light chain CDR2 of SEQ ID NO: 12; and a light chain CDR3 of SEQ ID NO: 13; and a heavy chain CDR1 of SEQ ID NO: 14; a heavy chain CDR2 of SEQ ID NO: 15; and a heavy chain CDR3 of SEQ ID NO: 16.

In another aspect, the present invention provides for the use of a MAdCAM-1 antibody for the manufacture of a medicament for the retreatment of non-alcoholic steatohepatitis (NASH).

In another aspect, the present invention provides a MAdCAM-1 antibody for use in treating non-alcoholic steatohepatitis (NASH).

In another aspect, the present invention provides a method for assessing the presence or absence of a beneficial response in a patient with non-alcoholic steatohepatitis (NASH) with different fibrosis stages after administering subcutaneously to the patient a dose of 22.5 mg or 75 mg of the MAdCAM-1 antibody, comprising: (a) measuring the level of a biomarker in a biological sample from said patient, wherein said biomarker is: (i) neoepitope-specific N-terminal pro-peptide of type III collagen (Pro-C3); and (ii) Enhanced Liver Fibrosis (ELF) Score; and (b) comparing said level with a control; wherein a change in the level of biomarker, as compared to the control, is predictive of a beneficial response in said patient.

In one aspect, a method for the treatment of a patient susceptible to or diagnosed with non-alcoholic steatohepatitis comprises administering to the patient a therapeutic dose of between 25 mg and 150 mg of a MAdCAM-1 antibody.

In some embodiments, the method comprises administering a subsequent dose of a MAdCAM-1 antibody.

In some embodiments, the method comprises either or both of the therapeutic dose and the subsequent dose of the MAdCAM-1 antibody is dosed at a range whose lower limit is 1 mg, 2 mg, 2.25 mg, 5 mg, 6 mg, 7 mg, 7.5 mg, 8 mg, 9 mg, 10 mg, 12 mg, 15 mg, 20 mg, 22.5 mg, 25 mg, 30 mg, 35 mg, 45 mg, 50 mg, 55 mg, 65 mg, 65 mg, 70 mg, 75 mg, 80 mg, 85 mg, 90 mg, 95 mg, 100 mg, 125 mg, 150 mg, 175 mg, 200 mg, or 225 mg and whose upper limit is 22.5 mg, 25 mg, 30 mg, 35 mg, 40 mg, 45 mg, 50 mg, 55 mg, 60 mg, 65 mg, 70 mg, less than 75 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, or 750 mg.

In some embodiments, the method comprises administering to the patient the therapeutic dose of 25 mg, 75 mg, or 150 mg of MAdCAM-1 antibody.

In some embodiments, the method comprises administering to the patient the therapeutic dose of 25 mg of MAdCAM-1 antibody.

In some embodiments, the method comprises administering to the patient the therapeutic dose of 75 mg of MAdCAM-1 antibody.

In some embodiments, the method comprises administering to the patient the therapeutic dose of 150 mg of MAdCAM-1 antibody.

In some embodiments, the subsequent dose is administered at an amount that is same or less than the therapeutic dose, and the subsequent dose is administered between about 1 week and about 12 weeks after the therapeutic dose.

In some embodiments, the subsequent dose is administered between about 1 week and about 24 weeks after the therapeutic dose.

In some embodiments, the subsequent dose is administered between about 1 week and about 52 weeks after the therapeutic dose.

In some embodiments, the subsequent dose is administered between about 1 week and about 72 weeks after the therapeutic dose.

In some embodiments, the subsequent dose is administered between about 1 week and about 104 weeks after the therapeutic dose.

In one aspect, a method for the treatment of a patient susceptible to or diagnosed with non-alcoholic steatohepatitis comprises administering to the patient a therapeutic dose of 25 mg, 75 mg, or 150 mg of a MAdCAM-1 antibody comprising a light chain CDR1 of SEQ ID NO: 11; a light chain CDR2 of SEQ ID NO: 12; and a light chain CDR3 of SEQ ID NO: 13; and a heavy chain CDR1 of SEQ ID NO: 14; a heavy chain CDR2 of SEQ ID NO: 15; and a heavy chain CDR3 of SEQ ID NO: 16.

In some embodiments, the method comprises administering to the patient the therapeutic dose of 25 mg of MAdCAM-1 antibody.

In some embodiments, the method comprises administering to the patient the therapeutic dose of 75 mg of MAdCAM-1 antibody.

In some embodiments, the method comprises administering to the patient the therapeutic dose of 150 mg of MAdCAM-1 antibody.

### DESCRIPTION OF THE DRAWING

FIG. 1 is a schematic representation of the study design showing all the three periods of the study—screening period, treatment period, and safety follow-up period. Different terms used in the schematics of the study design are elaborated herein.

The term “Screening” indicates the screening of the participants with NASH (Nonalcoholic Fatty Liver Disease Activity Score (NAS)≥4) and F4 fibrosis (i.e., cirrhosis). The screening period is extended up to 24 weeks until safety and tolerability information is available on four F2/F3 participants who have received at least 3 doses of the MAdCAM-1 antibody and have been monitored for at least 12 weeks.

The term “Liver biopsy” indicates that the eligibility for liver biopsy at Week −6 is established on the basis of results of Week −8 (SV1) assessments (Fibrosis-4 Index score (FIB-4)≥1.2, FibroScan-aspartate aminotransferase score (FAST)≥0.0.35, Pro-C3≥12.6 ng/mL, Enhanced Liver Fibrosis score (ELF)≥7.7, and liver chemistry eligibility criteria). For participants who meet other eligibility criteria at Week −8 (SV1), if ALT or AST is >4×ULN and <5×ULN at Week −8 (SV1), then the ALT/AST results at Week −6 (SV2) will also be evaluated before liver biopsy and abdominal MRI can be performed.

Pro-C3 and ELF Score are defined by an average of two measurements taken two weeks apart. Only participants with biopsy-proven NASH (NAS score≥4 with at least 1 point each in steatosis, ballooning, and lobular inflammation) and liver fibrosis stage 2, 3, or 4 are enrolled in the study. Qualifying historical liver biopsies are accepted if formalin-fixed paraffin-embedded (FFPE) liver blocks are available for further histological analyses and the participants meet the Pro-C3≥12.6 ng/ml and ELF≥7.7 inclusion criteria.

The term “Liver tests” indicates that the liver tests included are alanine aminotransferase (ALT), aspartate aminotransferase (AST), total bilirubin, alkaline phosphatase, gamma glutamyl transferase (GGT), and albumin.

The term “Biomarkers” indicates that the biomarkers included are neoepitope-specific N-terminal pro-peptide of type III collagen (Pro-C3), Enhanced Liver Fibrosis (ELF) Score, soluble MAdCAM-1 (sMAdCAM-1), high-sensitivity C reactive protein (hsCRP), IL-8, calprotectin (serum), circulatory T-cell signatures (e.g., Th17/Th1 vs Th2 ratios), CCR9 and CXCR3.

The abbreviated terms in the schematics of the study design are defined herein. CAP indicates Controlled Attenuation Parameter; F indicates fibrosis stage; hsCRP indicates high-sensitivity C-reactive protein; IL indicates interleukin; LSM indicates liver stiffness measurement; Pro-C3 indicates neoepitope-specific N-terminal pro-peptide of type III collagen; Q4W indicates every 4 weeks; and sMAdCAM-1 indicates soluble mucosal addressin cell adhesion molecule 1.

FIG. 2A depicts the MRI and Fibroscan imaging readouts from subjects affected with fibrosis (fibro-inflammation). FIG. 2B depicts the MRI and Fibroscan imaging readouts from subjects affected with steatosis (LSM=liver stiffness measurement; CAP=Controlled Attenuation Parameter; cT1=iron-corrected T1 mapping; PDFF=proton density fat fraction; LB=liver biopsy). FIG. 2C shows the MRI and Fibroscan imaging readouts from subjects affected with fibrosis (fibro-inflammation) as well as from subjects affected with steatosis.

### DEFINITIONS

In order for the present invention to be more readily understood, certain terms are first defined below. Additional definitions for the following terms and other terms are set forth throughout the specification.

As used in the specification and claims, the singular form “a”, “an”, and “the” include plural references unless the context clearly dictates otherwise. For example, the term “a cell” includes a plurality of cells, including mixtures thereof.

Antibody: As used herein, the term “antibody” or “Ab” or “Abs” or “mAbs” refers to immunoglobulin molecules and immunologically active portions of immunoglobulin (Ig) molecules, i.e., molecules that contain an antigen binding site that specifically binds (immunoreacts with) an antigen. By “specifically binds” or “immunoreacts with” it is meant that the antibody reacts with one or more regions of a desired antigen of the desired antigen. Antibodies include antibody fragments. Antibodies also include, but are not limited to, polyclonal, monoclonal, chimeric dAb (domain antibody), single chain, F_{ab}, F_{ab′}, F_{(ab′)2} fragments, scFvs, and F_{ab} expression libraries. An antibody may be a whole antibody, or immunoglobulin, or an antibody fragment.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH2-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

Antigen binding site: As used herein, the term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions”, are interposed between more conserved flanking stretches known as “framework regions” or “FRs.” Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Approximately or about: As used herein, the term “approximately” or “about” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Epitope: As used herein, the term “epitope” includes any protein determinant capable of specific binding to an immunoglobulin, or fragment. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” of a therapeutic agent means an amount that is sufficient, when administered to a patient suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose. In the context of therapeutic (including prophylactic) applications, the amount of active agent administered to the patient will depend on the type and severity of the disease or condition and on the characteristics of the patient, such as general health, age, sex, body weight and tolerance to drugs. It will also depend on the degree, severity and type of disease or condition. The skilled artisan will be able to determine appropriate dosages depending on these and other factors.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a patient who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g., 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise.

### ABBREVIATIONS

In order for the present invention to be more readily understood, certain abbreviations have been used, which are first defined below in Table 1. Additional abbreviations for the following terms and other terms are set forth throughout the specification.

TABLE 1
Abbreviations

| | |
|---|---|
| 6-MP | 6-mercaptopurine |
| AE | adverse event |
| ALP | alkaline phosphatase |
| ALT | alanine aminotransferase |
| AST | aspartate aminotransferase |
| AZA | azathioprine |
| β-hCG | beta-human chorionic gonadotropin |
| CAP | Controlled Attenuation Parameter |
| CD | Crohn's disease |
| CFR | Code of Federal Regulations |
| CI | confidence interval |
| CLD | chronic liver disease |
| CRF | case report form |
| CRO | contract research organization |
| CSR | clinical study report |
| cT1 | iron-corrected T1 |
| CTIS | Clinical Trial Information System |
| DMC | data monitoring committee |
| DSI | disease severity index |
| ECG | electrocardiogram |
| eConsent | electronic consent |
| eCRF | electronic case report form |
| EEA | European Economic Area |
| ELF | Enhanced Liver Fibrosis test |
| EMA | European Medicines Agency |
| EudraCT | European Union clinical trials database |
| F | fibrosis stage |
| FAST | FibroScan-aspartate aminotransferase |
| FDA | Food and Drug Administration (US) |
| FIB-4 | Fibrosis-4 Index |
| GCP | Good Clinical Practice |
| GGT | gamma-glutamyl transferase |
| GLP-1 RA | glucagon-like peptide-1 receptor agonist |
| HBsAg | hepatitis B virus surface antigen |
| HbA1c | Hemoglobin Alc |
| HCV | hepatitis C virus |
| HIV | human immunodeficiency virus |
| hsCRP | high-sensitivity C-reactive protein |
| IB | investigator's brochure |
| IBD | inflammatory bowel disease |
| ICF | informed consent form (including electronic |
| | consent where applicable) |
| ICH | International Council for Harmonisation of |
| | Technical Requirements for Registration of |
| | Pharmaceuticals for Human Use |
| IGRA | Interferon-gamma release assay |
| IMID | immune-mediated inflammatory disease |
| INR | international normalized ratio |
| IRB | institutional review board |
| IRT | Interactive Response Technology |
| LSM | liver stiffness measurement |
| MAdCAM-1 | mucosal addressin cell adhesion molecule 1 |
| MCD | methionine choline deficient |
| MELD | Model for End-stage Liver Disease |
| mmHg | millimeter(s) of mercury |
| MMRM | mixed effects model for repeated measure |
| MRI | magnetic resonance imaging |
| MTX | methotrexate |
| NAb | neutralizing antibody |
| NAFLD | nonalcoholic fatty liver disease |
| NAS | Nonalcoholic Fatty Liver Disease Activity Score |
| NASH | nonalcoholic steatohepatitis |
| PD | pharmacodynamic(s) |
| PDFF | proton density fat fraction |
| PIIINP | amino-terminal propeptide of type III procollagen |
| PK | pharmacokinetic(s) |
| PML | progressive multifocal leukoencephalopathy |
| Pro-C3 | neoepitope-specific N-terminal pro-peptide of |
| | type III collagen |
| PSC | primary sclerosing cholangitis |
| Q4W | every 4 weeks |
| RBC | red blood cell |
| RNA | ribonucleic acid |
| RSI | reference safety information |
| SAE | serious adverse event |
| SAP | statistical analysis plan |
| SC | subcutaneous(ly) |
| SoA | Schedule of Activities |
| SUSAR | suspected unexpected serious adverse reaction |
| TB | tuberculosis |
| TBL | total bilirubin |
| TE | transient elastography |
| TEAE | treatment-emergent adverse event |
| TIMP-1 | tissue inhibitor of metalloproteinases 1 |
| TNF | tumor necrosis factor |
| TZD | thiazolidinedione |
| UC | ulcerative colitis |
| ULN | upper limit of the normal range |
| US | United States |

### DETAILED DESCRIPTION

Embodiments of the present disclosure are described below. It is, however, expressly noted that the present disclosure is not limited to these embodiments, but rather the intention is that modifications that are apparent to the person skilled in the art and equivalents thereof are also included.

The present invention is directed to dosing regimen for administering a Mucosal Addressin Cell Adhesion Molecule-1 (MAdCAM-1; also known as addressin) antagonist antibody to patients with Non-alcoholic Steatohepatitis (NASH). In one aspect, the invention features a method for treating non-alcoholic steatohepatitis (NASH), the method comprising administering to the patient in need thereof an effective amount of a MAdCAM-1 antibody.

### Non-Alcoholic Fatty Liver Disease (NAFLD) and Non-Alcoholic Steatohepatitis (NASH)

Non-alcoholic fatty liver disease (NAFLD) has become one of the most prominent forms of chronic liver disease worldwide, mirroring the obesity epidemic. NAFLD is generally viewed as a spectrum of liver disease in which hepatic steatosis (or “fatty liver”), the accumulation of triglycerides in hepatocytes, develops in the absence of secondary causes (e.g., medications, excessive alcohol consumption, or certain heritable conditions).

Patients with NAFLD are at a high risk for cardiovascular morbidity and mortality (Chalasani et al., “The diagnosis and management of nonalcoholic fatty liver disease: Practice guidance from the American Association for the Study of Liver Diseases”, Hepatology, 67(1), 328-357 (2018). Nonalcoholic steatohepatitis (NASH) is the inflammatory subtype of NAFLD, with steatosis as well as evidence of hepatocyte injury (ballooning) and inflammation, with or without fibrosis (Chalasani et al. 2018). Although often clinically silent, NASH with fibrosis can progress to cirrhosis, end-stage liver disease, and in some patients, a need for a liver transplant. More than 20% of patients with NASH will develop cirrhosis in their lifetime (Matteoni et al., “Nonalcoholic fatty liver disease: a spectrum of clinical and pathological severity”, Gastroenterology, 116(6), 1413-1419 (1999)) and patients with NASH also have increased risk of developing hepatocellular carcinoma (Stine et al., “Systematic review with meta-analysis: risk of hepatocellular carcinoma in non-alcoholic steatohepatitis without cirrhosis compared to other liver diseases”, Aliment Pharmacol Ther, 48(7), 696-703 (2018)). From 2004 to 2016, there was a 114% and 80% expansion in liver transplant waitlist registration due to NASH for men and women, respectively (Noureddin et al., “NASH Leading Cause of Liver Transplant in Women: Updated Analysis of Indications For Liver Transplant and Ethnic and Gender Variances”, Am J Gastroenterol, 113(11), 1649-1659 (2018)), and NASH is now the leading indication for liver transplant listing for women and is expected to overtake alcoholic liver disease as the leading liver transplant indication for all patients within the next few years (Noureddin et al. 2018). NASH has an annual mortality 1.7 times higher than NAFLD overall (25.56 vs 15.44 events per 1000 person-years), and liver-specific mortality is 15 times higher than in NAFLD (11.77 vs 0.77 events per 1000 person-years) (Younossi et al., “Global epidemiology of nonalcoholic fatty liver disease—Meta-analytic assessment of prevalence, incidence, and outcomes”, Hepatology, 64(1), 73-84 (2016)).

Currently, the primary treatment for NAFLD/NASH is lifestyle modification for weight loss through diet and exercise (Guirguis et al., “Emerging therapies for the treatment of nonalcoholic steatohepatitis: A systematic review”, Pharmacotherapy, 41(3), 315-28 (2021); Sheka et al., “Nonalcoholic Steatohepatitis: A Review. Jama, 323(12), 1175-1183 (2020)). A meta-analysis studies showed the weight loss of 7% or greater was associated with improvement in the Nonalcoholic Fatty Liver Disease Activity Score (NAS) (Musso et al., “Impact of current treatments on liver disease, glucose metabolism and cardiovascular risk in non-alcoholic fatty liver disease (NAFLD): a systematic review and meta-analysis of randomised trials”, Diabetologia, 55(4), 885-904 (2012)). Although dietary composition does appear to have an effect on hepatic fat deposition, no specific macronutrient diet has been shown to have a benefit for NASH. Therefore, caloric restriction is the most appropriate recommendation for these patients (Chalasani et al. 2018).

Vitamin E and pioglitazone has shown some benefits in clinical studies. Pioglitazone, a peroxisome proliferator-activator receptor-gamma (PPAR-γ) agonist, has demonstrated improvement in insulin sensitivity, aminotransferase levels, steatosis, inflammation and ballooning in patients with NASH and prediabetes or type 2 diabetes mellitus (T2DM) (Belfort, “Metabolic and histologic improvement in non-alcoholic steatohepatitis (NASH) during pioglitazone (PIO) treatment is associated with a reduction in inflammatory markers”, Abstract No. 439-P, American Diabetes Association 66th Scientific Sessions, 9-13 Jun. 2006 Washington, DC. American Diabetes Association).

For patients with NASH, pharmacotherapy options are currently limited to off-label use of pioglitazone and vitamin E as large, randomized studies evaluating these therapies are lacking. To date, however, no pharmacotherapy is approved by the U.S. Food and Drug Administration (FDA) or the European Medicines Agency (EMA) for NASH.

### MAdCAM-1

MAdCAM-1 (also known as addressin) is a member of the immunoglobulin superfamily of cell adhesion receptors. The selectivity of lymphocyte homing to specialized lymphoid tissue and mucosal sites of the gastrointestinal tract is determined by the endothelial expression of MAdCAM-1. MAdCAM-1 is uniquely expressed on the cell surface of high endothelial venules of organized intestinal lymphoid tissue, such as Peyer's patches and mesenteric lymph nodes, but also in other lymphoid organs, such as pancreas, gall bladder and splenic venules and marginal sinus of the splenic white pulp.

While MAdCAM-1 plays a physiological role in gut immune surveillance, it appears to facilitate excessive lymphocyte extravasation in inflammatory conditions. Tumor necrosis factor alpha (TNFα) and other pro-inflammatory cytokines increase endothelial MAdCAM-1 expression and, in biopsy specimens taken from patients with Crohn's disease (CD) and ulcerative colitis (UC), there is an approximate 2-3 fold focal increase in MAdCAM-1 expression at sites of inflammation. Similar patterns of elevated expression have been observed in experimental models of colitis. In other pre-clinical models for inflammatory conditions, such as insulin-dependent diabetes graft versus host disease, chronic liver disease, inflammatory encephalopathy, and gastritis, there is also reawakening of fetal MAdCAM-1 expression and participation of activated α₄β₇⁺ lymphocytes in disease pathogenesis.

A recent publication (Graham et al., “Aberrant hepatic trafficking of gut-derived T cells is not specific to primary sclerosing cholangitis”, Hepatology, 75(3), 518-30 (2022)) confirmed MAdCAM-1 expression in explanted livers of patients undergoing orthotopic liver transplantation for chronic liver disease (CLD). Positive hepatic MAdCAM-1 immunoreactivity was described in more than 75% of patients with CLD regardless of etiology or disease severity and is largely universal in cases of later stages of CLD. There was increased expression of both CCL25 and MAdCAM-1, which can induce tissue infiltration of α4β7- and C-C chemokine Receptor Type 9 (CCR9)-expressing CD4⁺ T cells and a concomitant reduction in peripheral frequencies (Graham et al. 2022), across all CLD groups compared with normal liver. The authors also found an increase in the number of α4β7⁺ CD4⁺ T-effector memory cells in livers, an increase in E4β7⁺ CD8⁺ cells, and that these β7⁺ cells displayed an increased proinflammatory phenotype. Consistent with the work of Grant and colleagues (Grant et al., “MAdCAM-1 expressed in chronic inflammatory liver disease supports mucosal lymphocyte adhesion to hepatic endothelium (MAdCAM-1 in chronic inflammatory liver disease)”, Hepatology, 33(5), 1065-1072 (2001)), these findings were independent of the etiology of the liver disease.

Aberrant hepatic expression of MAdCAM-1 seems to be functional. The liver inflammatory lymphocytes of patients with PSC are mainly non-activated memory T-lymphocytes, a substantial proportion of which expressed the co-receptors α₄β₇ and/or CCR9(Ponsioen et al., “Immunohistochemical analysis of inflammation in primary sclerosing cholangitis”, Eur. J. Gastroenterol. Hepatol’, 11(7), 769-74 (1999)).

### Dosage Regiments

The present invention relates to dosing regimen for administering a MAdCAM-1 antibody to patients susceptible to or diagnosed with NASH. In one aspect, the invention provides a method for treating a patient susceptible to or diagnosed with NASH comprising administering to the patient a therapeutic dose of between about 20 mg and about 150 mg of a MAdCAM-1 antibody. In some embodiments the method further comprises administering a subsequence dose of a MAdCAM-1 antibody.

In some embodiments, the therapeutic dose of the MAdCAM-1 antibody is dosed at a range whose lower limit is 1 mg, 2 mg, 2.25 mg, 5 mg, 6 mg, 7 mg, 7.5 mg, 8 mg, 9 mg, 10 mg, 12 mg, 15 mg, 20 mg, 22.5 mg, 25 mg, 30 mg, 35 mg, 45 mg, 50 mg, 55 mg, 65 mg, 65 mg, 70 mg, 75 mg, 80 mg, 85 mg, 90 mg, 95 mg, 100 mg, 125 mg, 150 mg, 175 mg, 200 mg, or 225 mg and whose upper limit is 22.5 mg, 25 mg, 30 mg, 35 mg, 40 mg, 45 mg, 50 mg, 55 mg, 60 mg, 65 mg, 70 mg, less than 75 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, or 750 mg.

In some embodiments, the subsequent dose of the MAdCAM-1 antibody is dosed at a range whose lower limit is 1 mg, 2 mg, 2.25 mg, 5 mg, 6 mg, 7 mg, 7.5 mg, 8 mg, 9 mg, 10 mg, 12 mg, 15 mg, 20 mg, 22.5 mg, 25 mg, 30 mg, 35 mg, 45 mg, 50 mg, 55 mg, 65 mg, 65 mg, 70 mg, 75 mg, 80 mg, 85 mg, 90 mg, 95 mg, 100 mg, 125 mg, 150 mg, 175 mg, 200 mg, or 225 mg and whose upper limit is 22.5 mg, 25 mg, 30 mg, 35 mg, 40 mg, 45 mg, 50 mg, 55 mg, 60 mg, 65 mg, 70 mg, less than 75 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, or 750 mg.

In some embodiments, either or both of the therapeutic dose and the subsequent dose of the MAdCAM-1 antibody is dosed at a range between about 20 mg to about 125 mg. In some embodiments, either or both of the therapeutic dose and the subsequent dose of the MAdCAM-1 antibody is dosed at a range between about 22.5 mg to about 120 mg. In some embodiments, either or both of the therapeutic dose and the subsequent dose of the MAdCAM-1 antibody is dosed at a range between about 25 mg to about 150 mg. In some embodiments, either or both of the therapeutic dose and the subsequent dose of the MAdCAM-1 antibody is dosed at a range between about 25 mg to about 115 mg. In some embodiments, either or both of the therapeutic dose and the subsequent dose of the MAdCAM-1 antibody is dosed at a range between about 30 mg to about 110 mg. In some embodiments, either or both of the therapeutic dose and the subsequent dose of the MAdCAM-1 antibody is dosed at a range between about 35 mg to about 105 mg. In some embodiments, either or both of the therapeutic dose and the subsequent dose of the MAdCAM-1 antibody is dosed at a range between about 40 mg to about 100 mg. In some embodiments, either or both of the therapeutic dose and the subsequent dose of the MAdCAM-1 antibody is dosed at a range between about 45 mg to about 95 mg. In some embodiments, either or both of the therapeutic dose and the subsequent dose of the MAdCAM-1 antibody is dosed at a range between about 50 mg to about 90 mg. In some embodiments, either or both of the therapeutic dose and the subsequent dose of the MAdCAM-1 antibody is dosed at a range between about 55 mg to about 85 mg. In some embodiments, either or both of the therapeutic dose and the subsequent dose of the MAdCAM-1 antibody is dosed at a range between about 60 mg to about 80 mg. In some embodiments, either or both of the therapeutic dose and the subsequent dose of the MAdCAM-1 antibody is dosed at a range between about 65 mg to about 75 mg.

In some embodiments, either or both of the therapeutic dose and the subsequent dose of the MAdCAM-1 antibody is dosed at about 22.5 mg or about 75 mg.

In some embodiments, the therapeutic dose of the MAdCAM-1 antibody is dosed at about 22.5 mg or about 75 mg. In some embodiments, the therapeutic dose of the MAdCAM-1 antibody is dosed at about 22.5 mg. In some embodiments, the therapeutic dose of the MAdCAM-1 antibody is dosed at about 75 mg.

In some embodiments, the subsequent dose of the MAdCAM-1 antibody is dosed at about 22.5 mg or about 75 mg. In some embodiments, the subsequent dose of the MAdCAM-1 antibody is dosed at about 22.5 mg. In some embodiments, the subsequent dose of the MAdCAM-1 antibody is dosed at about 75 mg.

In some embodiments, either or both of the therapeutic dose and the subsequent dose of the MAdCAM-1 antibody is dosed at about 25 mg or about 75 mg or about 150 mg.

In some embodiments, either or both of the therapeutic dose and the subsequent dose of the MAdCAM-1 antibody is dosed at about 25 mg.

In some embodiments, either or both of the therapeutic dose and the subsequent dose of the MAdCAM-1 antibody is dosed at about 75 mg.

In some embodiments, either or both of the therapeutic dose and the subsequent dose of the MAdCAM-1 antibody is dosed at about 150 mg.

In some embodiments, the therapeutic dose of the MAdCAM-1 antibody is dosed at about 25 mg or about 75 mg or about 150 mg.

In some embodiments, the therapeutic dose of the MAdCAM-1 antibody is dosed at about 25 mg.

In some embodiments, the therapeutic dose of the MAdCAM-1 antibody is dosed at about 75 mg.

In some embodiments, the therapeutic dose of the MAdCAM-1 antibody is dosed at about 150 mg.

In some embodiments, the subsequent dose of the MAdCAM-1 antibody is dosed at about 25 mg.

In some embodiments, the subsequent dose of the MAdCAM-1 antibody is dosed at about 75 mg.

In some embodiments, the subsequent dose of the MAdCAM-1 antibody is dosed at about 150 mg.

In some embodiments, the methods of the invention further provide for the administration of subsequent doses of the antibody in an amount that is approximately the same or less than the initial dose.

In some embodiments, either or both of the therapeutic dose and the subsequent dose of the MAdCAM-1 antibody is administered at doses of 0.03, 0.1, 0.3, 0.33, 1.0, 2.0, 3.0 or 10 mg/kg of the patient's body weight. In one study phase, patients receive intravenous administration of the MAdCAM-1 antibody at doses of 0.03, 0.1, 0.3, 0.33, 1.0, 0.2, 3.0 or 10 mg/kg of the patient's body weight. In another study phase, patients receive subcutaneous administration of the MAdCAM-1 antibody at doses of 0.03, 0.1, 0.3, 0.33, 1.0, 2.0, 3.0 or 10 mg/kg of the patient's body weight. In one phase study, the MAdCAM-1 antibody is administered intravenously at the following doses: 0.03, 0.1, 0.3, 0.33, 1, 2, and 10 mg/kg of the patient's body weight.

### Dosage Interval

In some embodiments, a subsequent dose is administered between about 1 and about 12 weeks after therapeutic dose. In some embodiments, a subsequent dose is administered between about 1 and about 52 weeks after therapeutic dose. In some embodiments, a subsequent dose is administered between about 1 and about 72 weeks after therapeutic dose. In some embodiments, a subsequent dose is administered between about 1 and about 104 weeks after therapeutic dose. In some embodiments, a subsequent dose is administered up to 3-5 years after therapeutic dose. In some embodiments, a subsequent dose is administered up to 3-5 years. In some embodiments, the subsequent dose is provided about 4 weeks after the therapeutic dose.

In some embodiments, the subsequent dose is administered between about 4 and about 12 weeks after the therapeutic dose. In some embodiments, the subsequent dose is administered between about 2 and about 8 weeks after the therapeutic dose. In some embodiments, the subsequent dose is administered between about 2 and about 10 weeks after the therapeutic dose. In some embodiments, the subsequent dose is administered between about 4 and about 10 weeks after the therapeutic dose. In some embodiments, the subsequent dose is administered given about 4 weeks after the therapeutic dose.

In some embodiments, the subsequent dose is administered between about 1 and about 3 months after the therapeutic dose. In some embodiments, the subsequent dose is administered about 1 month apart. In some embodiments, the subsequent dose is administered about 2 months after the therapeutic dose.

In some embodiments, the MAdCAM-1 antibody is administered every 2 weeks for 52 weeks. In some embodiments, the MAdCAM-1 antibody is administered every 4 weeks for 52 weeks. In some embodiments, the MAdCAM-1 antibody is administered every 6 weeks for 52 weeks.

In some embodiments, the MAdCAM-1 antibody is administered every 2 weeks for 72 weeks. In some embodiments, the MAdCAM-1 antibody is administered every 4 weeks for 72 weeks. In some embodiments, the MAdCAM-1 antibody is administered every 6 weeks for 72 weeks.

In some embodiments, the MAdCAM-1 antibody is administered every 2 weeks for 104 weeks. In some embodiments, the MAdCAM-1 antibody is administered every 4 weeks for 104 weeks. In some embodiments, the MAdCAM-1 antibody is administered every 6 weeks for 104 weeks.

In some embodiments, the MAdCAM-1 antibody is administered every 2 weeks for 3-5 years. In some embodiments, the MAdCAM-1 antibody is administered every 4 weeks for 3-5 year. In some embodiments, the MAdCAM-1 antibody is administered every 6 weeks for 3-5 years.

In some embodiments, the MAdCAM-1 antibody is administered every 2 weeks indefinitely. In some embodiments, the MAdCAM-1 antibody is administered every 4 weeks indefinitely. In some embodiments, the MAdCAM-1 antibody is administered every 6 weeks indefinitely.

### MAdCAM-1 Antibodies

The invention relates to MAdCAM-1 antibodies in general and their use in treating a patient susceptible to or diagnosed with NASH. The MAdCAM-1 antibodies include, but not limited to, the antibodies that are listed in Table 2, below.

TABLE 2
MAdCAM-1 antibodies

| | Light Chain | Heavy Chain |
|---|---|---|
| Antibody | SEQ ID NO: | SEQ ID NO: |
| 1.7.2 | 17 | 18 |
| 1.8.2 | 19 | 20 |
| 6.14.2 | 21 | 22 |
| 6.22.2 | 23 | 24 |
| 6.34.2 | 25 | 26 |
| 6.67.1 | 27 | 28 |
| 6.73.2 | 29 | 30 |
| 6.77.1 | 31 | 32 |
| 7.16.6 | 1 | 2 |
| 7.20.5 | 33 | 34 |
| 7.26.4 | 35 | 36 |
| 9.8.2 | 37 | 38 |

TABLE 3
MAdCAM-1 antibodies heavy chain
and light chain amino acid sequences

DIVMTQTPLSLSVTPGQPASISCKSSQSLLHTDGTTYLYWYLQKPGQPPQLLIYEVSN
RFSGVPDRFSGSGSGTDFTLKISRVEAEDVGIYYCMQNIQLPWTFGQGTKVEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
(SEQ ID NO: 1)
QVQLVQSGAEVKKPGASVKVSCKASGYTFTSYGINWVRQAPGQGLEWMGWISVYS
GNTNYAQKVQGRVTMTADTSTSTAYMDLRSLRSDDTAVYYCAREGSSSSGDYYYG
MDVWGQGTTVTVSSASTKGPSVFPLAPCSRSTSESTAALGCLVKDYFPEPVTVSWNS
GALTSGVHTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWESNGQPENNY
KTTPPMLDSDGSFFLYSKLTVDKSRWQQGNVFSCSVMHEALHNHYTQKSLSLSPGK
(SEQ ID NO: 2)
DIVMTQSPLSLPVTPGEPASISCRSSQSLLQSNGYNYLDWYLQKPGQSPQLLIYLGSN
RASGVPDRFSGSGSGTDFTLKISRVEAEDVGVYYCMQALQTITFGQGTRLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
(SEQ ID NO: 17)
EVQLVESGGGLVKPGGSLRLSCVASGFTFTNAWMIWVRQAPGKGLEWVGRIKRKT
DGGTTDYAAPVKGRFTISRDDSKNTLYLQMNSLKTEDTAVYYCTTGGVAEDYWGQ
GTLVTVSSASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVERKCCVECP
PCPAPPVAGPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPPML
DSDGSFFLYSKLTVDKSRWQQGNVFSCSVMHEALHNHYTQKSLSLSPGK
(SEQ ID NO: 18)
DIVMTQSPLSLPVTPGEPASISCRSSQSLLQSNGFNYLDWYLQKPGQSPQLLIYLGSNR
ASGVPDRFSGSGSGTDFTLKISRVEAEDVGVYYCMQALQTITFGQGTRLEIKRTVAA
PSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
(SEQ ID NO: 19)
EVQLVESGGGLVKPGGSLRLSCVVSGFTFTNAWMIWVRQAPGKGLEWVGRIKRKT
DGGTTDYAAPVKGRFTISRDDSKNTLYLQMNSLKTEDTAVYYCTTGGVAEDYWGQ
GTLVTVSSASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVERKCCVECP
PCPAPPVAGPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPPML
DSDGSFFLYSKLTVDKSRWQQGNVFSCSVMHEALHNHYTQKSLSLSPGK
(SEQ ID NO: 20)
DIQMTQSPSSLSASVGDRVTITCRASRSISSYLNWYQQKPGKAPKVLIFFVSSLQSGVP
SRFSGSGSGTDFTLTISSLQPEDFATYYCQQNYIPPITFGQGTRLEIRRTVAAPSVFIFPP
SDEQLKSGTASVVCLLNNFYPREAKVQWKVDNALQSGNSQESVTEQDSKDSTYSLS
STLTLSKADYEKHKVYACEVTHQGLSSPVTKSFNRGEC
(SEQ ID NO: 21)
EVQLLESGGGLVQPGGSLRLSCAASGLTFNNSAMTWVRQAPGKGLEWVSTTSGSGG
TTYYADSVKGRFTISRDSPKNTLYLQMNSLRAEDTAVYYCAARGYSYGTTPYEYWG
QGTLVTVSSASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRVESKYGPPC
PSCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK
(SEQ ID NO: 22)
EIVLTQSPDFQSVTPKEKVTITCRASQRIGSSLHWYQQKPDQSPKLLIKYASQSFSGVP
SRFSGSGSGTNFTLTINGLEAEDAATYYCHQSGRLPLTFGGGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
(SEQ ID NO: 23)
QVQLVESGGGVVQPGRSLRLSCAASGHTFSSDGMHWVRQAPGKGLEWVAIIWYDG
SNKYYADSVKGRFTISRDNSKNTLYLQMNSLRAEDTAVYYCARDPGYYYGMDVW
GQGTTVTVSSASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDKRVESKYGPP
CPSCPAPEFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTAPP
VLDSDGSFFLYSRLTVDKSRWQEGNVFSCSVMHEALHNHYTQKSLSLSLGK
(SEQ ID NO: 24)
DIQMTQSPSSLSASVGDRVTITCRASQNISSYLNWFQQKPGKAPKLLIYAASGLKRGV
PSRFSGSGSGTDFTLTIRTLQPDDFATYSCHQSYSLPFTFGPGTKVDIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
(SEQ ID NO: 25)
QVQLVESGGGVVQPGRSLRLSCAASGFTFSSYGMHWVRQAPGKGLEWVAVISNDG
NNKYYADSVKGRFTISRDNSKNTLYLQMNSLSAEDTAVYYCARDSTAITYYYYGM
DVWGQGTTVTVSSASTKGPSVFPLAPCSRSTSESTAALGCLVKDYFPEPVTVSWNSG
ALTSGVHTFPAVLQSSGLYSLSSVVTVPSSSLGTKTYTCNVDHKPSNTKVDKRVESK
YGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNKGLPSSIEKT
ISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK
(SEQ ID NO: 26)
DIVMTQSPDSLAVSLGERATINCKSSQSVLYSSNNKTYLAWYQQKPRQPPKLLIYWA
SIREYGVPDRFSGSGSGTDFTLTISSLQAEDVAVYFCQQYYSIPPLTFGGGTKVEIKRT
VAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
(SEQ ID NO: 27)
QVQLQESGPGLVKPSETLSLTCTVSGDSISSNYWSWIRQPAGKGLEWIGRIYTSGGTN
SNPSLRGRVTILADTSKNQFSLKLSSVTAADTAVYYCARDRITIIRGLIPSFFDYWGQG
TLVTVSSASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKYGPPCPSC
PAPEFLGGPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
(SEQ ID NO: 28)
DIQMTQSPSSLSASVGDRVTFTCRASQNITNYLNWYQQKPGKAPKLLIYAASSLPRG
VPSRFRGSGSGTDFTLTISSLQPEDFATYYCQQSYSNPPECGFGQGTTLDIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
(SEQ ID NO: 29)
EVQLLESGGDLVQPGGSLRLSCAASGFTFRSYAMNWVRQAPGKGLEWVSVISGRGG
TTYYADSVKGRFTISRDNSKNTLYLQMNSLRAEDAAVYYCAKIAVAGEGLYYYYG
MDVWGQGTTVTVSSASTKGPSVFPLAPCSRSTSENTAALGCLVKDYFPEPVTVSWN
SGALTSGVHTFPAVLQSSGLYSLSSVVTVPSSSLGTKTYTCNVDHKPSNTKVDKRVE
SKYGPPCPSCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLGK
(SEQ ID NO: 30)
DIVMTQTPLSLSVTPGQPASISCNSSQSLLLSDGKTYLNWYLQKPGQPPQLLIYEVSN
RFSGVPDRFSGSGSGTDFTLKISRVEAEDVGVYSCMQSIQLMCSFGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
(SEQ ID NO: 31)
EVQLVESGGGLVKPGGSLRLSCAASGFTFSSYSMNWVRQAPGKGLEWVSSISSSSSYI
YYADSVKGRFTISRDNAKNSLYLQMNSLRAEDTAVYYCARDGYSSGWSYYYYYG
MDVWGQGTTVTVSSASTKGPSVFPLAPCSRSTSESTAALGCLVKDYFPEPVTVSWNS
GALTSGVHTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPENNY
KTTPPVLDSDGSFFLYSRLTVDKSRWQEGNVFSRSVMHEALHNHYTQKSLSLSLGK
(SEQ ID NO: 32)
DIVMTQSPLSLPVTPGEPASISCRSSQSLLHGNGYNYLDWYLQKPGQSPQLLIYLGSN
RASGVPDRFSGSGSGTDFTLKISRVEAEDVGVYYCMQALQTLTFGGGTKVEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
(SEQ ID NO: 33)
QVQLQESGPGLVKPSETLSLTCTVSGSSISSYHWNWIRQPAGKGLEWIGRIYTSGSTN
YNPSLKSRVTMSLDTSKNQFSLKLSSVTAADTAVYYCAREGVRYYYASGSYYYGLD
VWGQGTTVTVSSASTKGPSVFPLAPCSRSTSESTAALGCLVKDYFPEPVTVSWNSGA
LTSGVHTFPAVLQSSGLYSLSSVVTVPSSNFGTQTYTCNVDHKPSNTKVDKTVERKC
CVECPPCPAPPVAGPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLPAPIEKTI
SKTKGQPREPQVYTLPPSREEMTKNQVSLTCLVKGFYPSDIAVEWESNGQPENNYKT
TPPMLDSDGSFFLYSKLTVDKSRWQQGNVFSCSVMHEALHNHYTQKSLSLSPGK
(SEQ ID NO: 34)
DIVMTQTPLSLSVTPGQPASISCKSNQSLLYSDGKTYLFWYLQKPGQPPQLLIYEVSN
RFSGVPDRFSGSGSGTDFTLKISRVEAEDVGVYYCMQSIQLPWTFGQGTKVEIKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
(SEQ ID NO: 35)
QVQLVQSGAEVKKPGASVKVSCEASGYTFTSYGIDWVRQAPGQGLEWMGWISVYS
GNTNYAQKLQGRVTMSTDTSTSTAYMELRSLRSDDTAVYYCAREGSSSSGDYYYG
MDVWGQGTTVTVSSASTKGPSVFPLAPCSRSTSESTAALGCLVKDYFPEPVTVSWNS
GALTSGVHTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWESNGQPENNY
KTTPPMLDSDGSFFLYSKLTVDKSRWQQGNVFSCSVMHEALHNHYTQKSLSLSPGK
(SEQ ID NO: 36)
DIQMTQSPSSLSASVGDRVTITCQASQDISNYLNWYQQKPGKAPKLLIYDASNLETG
VPSRFSGSGSGTDFTFTISSLQPEDIATYSCQHSDNLSITFGQGTRLEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
(SEQ ID NO: 37)
QVQLVESGGGVVQPGRSLRLSCAASGFTFSSYGMHWVRQAPGKGLEWVAVIWYDG
SNEYYADSVKGRFTISRDNSKNTLYLQMNSLRAEDTAVYYCARGAYHFAYWGQGT
LVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPSCP
APEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
(SEQ ID NO: 38)

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ECACC under the terms of the Budapest Treaty, and subject to an agreement between Pfizer Inc. and ECACC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. § 122 and the Commissioner's rules pursuant thereto (including 37 C.F.R. § 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws. Therapeutic methods of the invention

In some embodiments, the antibody is mAb 7.16.6 (listed in Table 2), or a variant thereof. In some embodiments, the MAdCAM-1 antibody comprises the CDRs of SEQ ID NO:1 and SEQ ID NO:2. In some embodiments, the MAdCAM-1 antibody comprises SEQ ID NO:1 and SEQ ID NO:2. In some embodiments, the MAdCAM-1 antibody comprises the CDRs of SEQ ID NO:3 and SEQ ID NO:4. In some embodiments, the MAdCAM-1 antibody comprises the variable domains of SEQ ID NO:3 and SEQ ID NO:4, which are further characterized in Table 4 provided herein, below.

TABLE 4
mAb 7.16.6 VH, VL, and CDR Sequences

| SEQ ID | Description | Sequence |
|---|---|---|
| 3 | mAb 7.16.6 VL | DIVMTQTPLSLSVTPGQPA |
| | CDRs underlined | SISCKSSQSLLHTDGTTYL |
| | SEQ IDs 11, 12, | YWYLQKPGQPPQLLIYEVS |
| | 13 | NRFSGVPDRFSGSGSGTDF |
| | | TLKISRVEAEDVGIYYCMQ |
| | | NIQLPWTFGQGTKVEIK |
| 4 | mAb 7.16.6 VH | QVQLVQSGAEVKKPGASVK |
| | CDRs underlined | VSCKASGYTFTSYGINWVR |
| | SEQ IDs 14, 15, | QAPGQGLEWMGWISVYSGN |
| | 16 | TNYAQKVQGRVTMTADTST |
| | | STAYMDLRSLRSDDTAVYY |
| | | CAREGSSSSGDYYYGMDVW |
| | | GQGTTVTVSS |
| 11 | mAb 7.16.6 VL, | KSSQSLLHTDGTTYLY |
| | CDR1 | |
| 12 | mAb 7.16.6 VL, | EVSNRFS |
| | CDR2 | |
| 13 | mAb 7.16.6 VL, | MQNIQLPWT |
| | CDR3 | |
| 14 | mAb 7.16.6 VH, | SYGIN |
| | CDR1 | |
| 15 | mAb 7.16.6 VH, | WISVYSGNTNYAQKVQG |
| | CDR2 | |
| 16 | mAb 7.16.6 VH, | EGSSSSGDYYYGMDV |
| | CDR3 | |

The MAdCAM-1 antibodies listed herein in Table 2, Table 3, and Table 4 are also described in WO 2016/110806 (herein incorporated by reference). Methods of making the MAdCAM-1 antibodies and their further characterizations, among other details, are provided in WO 2005/067620 and WO 2019/014572 (herein incorporated by reference).

In some embodiments, alternative MAdCAM-1 antibodies for use in methods and compositions of the invention may be used.

Exemplary MAdCAM-1 antibodies are listed in Table 1 and 2 of WO 2005/067620 (herein incorporated by reference). The examples of WO 2005/067620 describe fully the antibodies that are listed in Tables 1 and 2 of that international patent publication, and provide details of characterizing information such as binding affinities, Kₒₙ, K_{off}, K_{d}, and so on.

In some embodiments, the antibody may be a MAdCAM-1 antibody that cross competes with an antibody comprising SEQ ID NO:1 and 2.

In some embodiments, the antibodies of the invention have one or more of the following characteristics:
a half-life in human patients of between 20 and 60 days;
subcutaneous (SC) bioavailability of at least 50%; and/or
a K_{d} of ≤10 nM.

In some embodiments, the antibody may have a half-life in human patients of at least 30 days. In some embodiments, the antibody may have a half-life in human patients of at least 35 days. In some embodiments, the antibody may have a half-life in human patients of at least 40 days. In some embodiments, the antibody may have a half-life in human patients of at least 45 days. In some aspects, the antibody may have a half-life in human patients of at least 50 days.

In some embodiments, the antibody's SC bioavailability may be at least 60%. In some embodiments, the antibody's SC bioavailability may be at least 65%. In some embodiments, the antibody's SC bioavailability may be at least 70%. In some embodiments, the antibody's SC bioavailability may be at least 75%. In some embodiments, the antibody's SC bioavailability may be at least 80%. In some embodiments, the antibody's SC bioavailability may be at least 85%. In some embodiments, the antibody's SC bioavailability may be at least 90%. In some embodiments, the antibody's SC bioavailability may be at least 95%. In some embodiments, the antibody's SC bioavailability may be at least 90%. In some embodiments, the antibody's SC bioavailability may be at least 99%.

In some embodiments, the KD is measured by surface plasmon resonance. In some embodiments, surface plasmon resonance may be measured using a Biocore. In some embodiments, the SPR may be measured using Biacore with captured antibody and solution phase MAdCAM-1.

In some embodiments, the antibody has a K_{d} of ≤10 nM. In some embodiments, the antibody has a K_{d} of ≤1 nM. In some embodiments, the antibody has a K_{d} of ≤500 pM. In some embodiments, the antibody has a K_{d} of ≤200 pM. In some embodiments, the antibody has a K_{d} of ≤100 pM. In some aspects, the antibody has a K_{d} of ≤50 pM. In some embodiments, the antibody has a K_{d} of ≤20 pM. In some aspects, the antibody has a K_{d} of ≤10 pM. In some embodiments, the antibody has a K_{d} of ≤5 pM.

### Pharmaceutical Compositions and Administration

MAdCAM-1 antibodies and antigen-binding portions thereof can be incorporated into pharmaceutical compositions suitable for administration to a patient. Typically, the pharmaceutical composition comprises a MAdCAM-1 antibody or antigen-binding portion thereof and a pharmaceutically acceptable carrier. As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application. Typical compositions are in the form of injectable or infusible solutions, such as compositions similar to those used for passive immunization of humans. In one case the mode of administration is parenteral (e.g., intravenous, subcutaneous, intraperitoneal, intramuscular). In another case, the antibody is administered by intravenous infusion or injection. In another case, the antibody is administered by intramuscular or subcutaneous injection. Formulations for injection may be presented in unit dosage form, e.g., in ampoules or in multi-dose containers, with or without an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. Sterile injectable solutions can be prepared by incorporating the MAdCAM-1 antibody in the required amount in an appropriate solvent with one or a combination of ingredients enumerated above, as required, followed by filter sterilization.

Dosage values may vary with the type and severity of the condition to be alleviated. In the case of sterile powders for the preparation of sterile injectable solutions, the suitable methods of preparation include vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain cases, the MAdCAM-1 antibody compositions may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations may be used. See, e.g., Sustained and Controlled Release Drug Delivery Systems, J. R. Robinson, ed., Marcel Dekker, Inc., New York, 1978, which is incorporated herein by reference.

Additional active compounds also can be incorporated into the compositions (including any one or more of the previously disclosed additional therapeutic agents). In some cases, an inhibitory MAdCAM-1 antibody is co-formulated with and/or co-administered with one or more additional therapeutic agents. These agents include, without limitation, antibodies that bind other targets, anti-tumor agents, anti-angiogenesis agents, signal transduction inhibitors, anti-proliferative agents, chemotherapeutic agents, or peptide analogues that inhibit MAdCAM-1. Such combination therapies may require lower dosages of the inhibitory MAdCAM-1 antibody as well as the co-administered agents, thus avoiding possible toxicities or complications associated with the various monotherapies.

In some embodiments, the patent is not taking a tumor necrosis factor (TNF) antagonist or TNF inhibitor.

The compositions may include a “therapeutically effective amount” or a “prophylactically effective amount” of an antibody or antigen-binding portion. A “therapeutically effective amount” refers to an amount effective, at dosages and for periods of time necessary, to achieve the desired therapeutic result. A therapeutically effective amount of the antibody or antigen-binding portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antigen-binding portion are outweighed by the therapeutically beneficial effects. A “prophylactically effective amount” refers to an amount effective, at dosages and for periods of time necessary, to achieve the desired prophylactic result. Typically, since a prophylactic dose is used in patients prior to or at an earlier stage of disease, the prophylactically effective amount may be less than the therapeutically effective amount.

In some embodiments the MAdCAM-1 antibody is administered in a formulation as a sterile aqueous solution having a pH that ranges from about 5.0 to about 6.5, from about 1 mM to about 100 mM of histidine buffer, from about 0.01 mg/mL to about 10 mg/mL of polysorbate 80 or polysorbate 20, from about 100 mM to about 400 mM of a non-reducing sugar selected from but not limited to trehalose or sucrose, from about 0.01 mM to about 1.0 mM of disodium EDTA dihydrate and optionally comprise a pharmaceutically acceptable antioxidant in addition to a chelating agent. Suitable antioxidants include, but are not limited to, methionine, sodium thiosulfate, catalase, and platinum.

In some embodiments, the formulation is as set forth in WO 2006/096490, the contents of which are hereby incorporated. In some embodiments, the formulation comprises 75 mg/mL antibody, 10 mM histidine pH 5.5, 90 mg/mL trehalose, dihydrate, 0.1 mg/mL disodium EDTA dihydrate, and 0.4 mg/mL polysorbate 80.

In another embodiements, the formulation comprises: 20 mM histidine, 7% Trehalose, 0.4 mg/mL polysorbate 80, 0.1 mg/mL EDTA, pH 5.5, and 25 mg/mL and 75 mg/mL anti-MAdCAM-1 antibody.

In some embodiments, the present invention provides a method for treating a patient susceptible to or diagnosed with NASH comprising administering to the patient a therapeutic dose of a MAdCAM-1 antibody, wherein the MAdCAM-1 antibody is administered to the patient subcutaneously.

In some embodiments, the present invention provides a method for treating a patient susceptible to or diagnosed with NASH comprising administering to the patient a therapeutic dose of a MAdCAM-1 antibody, wherein the MAdCAM-1 antibody is administered to the patient intravenously.

The MAdCAM-1 antibody may also be administered continuously via a minipump. The MAdCAM-1 antibody may be administered via a mucosal, buccal, intranasal, inhalable, intravenous, subcutaneous, intramuscular, parenteral, or intratumor route. The MAdCAM-1 antibody may be administered once, at least twice or for at least the period of time until the condition is treated, palliated or cured. The MAdCAM-1 antibody generally will be administered for as long as the condition is present.

In one aspect, the present invention provides for the use of a MAdCAM-1 antibody for the manufacture of a medicament for the retreatment of NASH.

In one aspect, the present invention provides a MAdCAM-1 antibody for use in treating NASH.

### NASH Diagnosis and Evaluation

In one aspect, the present invention provides a method for assessing the presence or absence of a beneficial response in a patient with non-alcoholic steatohepatitis (NASH) with different fibrosis stages after administering subcutaneously to the patient a dose of 22.5 mg, 25 mg, 75 mg, or 150 mg of the MAdCAM-1 antibody, comprising: (a) measuring the level of a biomarker in a biological sample from said patient, wherein said biomarker is: (i) neoepitope-specific N-terminal pro-peptide of type III collagen (Pro-C3); and (ii) Enhanced Liver Fibrosis (ELF); and (b) comparing said level with a control; wherein a change in the level of biomarker, as compared to the control, is predictive of a beneficial response in said patient.

In some embodiments, NASH can be diagnosed by liver biopsy (e.g., histological evidence of steatosis, inflammation, and hepatocyte ballooning, for example in the absence of other causes of liver disease or substantial alcohol consumption). For example, the NAFLD Activity Score (NAS) can be useful in identifying patients with NASH. See Kleiner et al, “Design and Validation of a Histological Scoring System for Nonalcoholic Fatty Liver Disease”, Hepatology, 41(6): 1313-1321 (2005).

NAS is the sum of separate scores for steatosis (0-3), hepatocellular ballooning (0-2), and lobular inflammation (0-3), with a maximal score of 8. A NAS score can be generated upon biopsy according the criteria set forth in Kleiner et al., supra. See also Table 5.

TABLE 5
Components of NAFLD Activity Score (NAS)

| Component | Score | Description |
|---|---|---|
| Steatosis¹ | 0 | <5% |
| | 1 | 5-33% |
| | 2 | >33-66% |
| | 3 | >66% |
| Lobular | 0 | No foci |
| Inflammation² | 1 | <2 foci/200x |
| | 2 | 2-4 foci/200x |
| | 3 | >4 foci/200x |
| Hepatocyte | 0 | None |
| Ballooning | 1 | Few balloon cells³ |
| | 2 | Many balloon cells/prominent ballooning⁴ |

¹Steatosis score reflects amount of surface area involved by steatosis.
²Excludes acidophil bodies and portal inflammation
³Rare but definite ballooned hepatocytes or diagnostically borderline cases
⁴May occur with Mallory's hyalin

In some embodiments, a patient is selected based on a liver biopsy (e.g., a liver biopsy used to determine a NAS score).

In some embodiments, a patient is selected based on a NAS score. In some embodiments, the presence of NASH is established by a NAS score of greater than or equal to 2 and less than or equal to 3 (i.e., a NAS score that is 2-3). In some embodiments, the presence of NASH is established by a NAS score of 4 or more (i.e., a NAS score that is ≥4). In some embodiments, the presence of NASH may be established by a liver biopsy revealing a NAS score of 5 or more (i.e., a NAS score that is ≥5).

In some embodiments, a patient is selected based on a NAS score determined prior to treatment.

In some embodiments, the presence of NASH is established by a NAS score of greater than or equal to 2 and less than or equal to 3 (i.e., a NAS score that is 2-3). In some embodiments, the presence of NASH is established by a NAS score prior to treatment of 4 or more (i.e., a NAS score that is ≥4). In some embodiments, the presence of NASH may be established by a liver biopsy revealing a NAS score prior to treatment of 5 or more (i.e., a NAS score that is ≥5).

In some embodiments, NASH can be characterized by a NAFLD Activity Score (NAS) of 5 or more, where NAS is the sum of separate scores for steatosis (range: 0-3), hepatocellular ballooning (range: 0-2), and lobular inflammation (range: 0-3). See Kleiner et al., supra. Steatosis is the abnormal retention of lipids within the liver. The steatosis score represents the percent of hepatocytes containing fat droplets (steatosis) as 0 (<5%), 1 (5-33%), 2 (33-66%), and 3 (>66%). Patients treated for NASH according to the present disclosure can have a NAS steatosis score of 1, 2 or 3. Hepatocyte ballooning is a type of cell death visually characterized by hypertrophy and localization of cellular nuclei at or near the center of the cell. In some embodiments, hepatocyte ballooning is scored as 0 (none), 1 (few), or 2 (many cells with prominent ballooning). In some embodiments, lobular inflammation is scored according to the number of foci of inflammation: 0 (no foci), 1(<2 foci/200× field), and 2 (2-4 foci/200× field). In some embodiments, a patient has a lobular inflammation scores of 0, 1, 2 or 3, and a ballooning score of 0, 1 or 2, provided that the sum of the lobular inflammation score and the ballooning score is at least 2.

In some embodiments, methods and uses described herein comprise an improvement in NAS score. In some embodiments, the improvement occurs without worsening of fibrosis.

In some embodiments, administering of the MAdCAM-1 antibody results in a NAFLD Activity Score (NAS) of <4.

In some embodiments, methods and uses described herein result in a decrease of at least one (≥1) in the patient's NAS score.

In some embodiments, methods and uses described herein result in a decrease of at least two (≥2) in the patient's NAS score.

In some embodiments, methods and uses described herein result in a decrease of at least three (≥3) in the patient's NAS score.

In some embodiments, methods and uses described herein result in a decrease of one (1) to three (3) in the patient's liver steatosis score.

In some embodiments, methods and uses described herein result in a decrease of one (1) or two (2) in the patient's hepatocellular ballooning score.

In some embodiments, methods and uses described herein result in a decrease of one (1) to three (3) in the patient's lobular inflammation score.

In some embodiments, a patient has a steatosis score of 1, 2, or 3.

In some embodiments, steatosis comprises macrovesicular steatosis. In some embodiments, steatosis comprises microvesicular steatosis. In some embodiments, steatosis comprises macrovesicular and microvesicular steatosis.

In some embodiments, methods and uses described herein result in resolution of steatohepatitis without worsening of fibrosis in a patient in need thereof. In some embodiments, resolution comprises absence of hepatocellular ballooning (e.g., a ballooning score of 0); absent or mild inflammation (e.g., a ballooning score of 0-1); and/or with steatosis present or absent (e.g., a steatosis score of 0-3).

In some embodiments, NASH is steatohepatitis characterized by at least one of lobular inflammation and hepatocyte ballooning. In some embodiments, NASH occurs in the absence of other causes of liver disease and/or substantial alcohol consumption.

In some embodiments, a patient has liver inflammation. In some embodiments, liver inflammation is lobular inflammation.

In some embodiments, administering of the MAdCAM-1 antibody results in a decrease in liver inflammation.

In some embodiments, administering of the MAdCAM-1 antibody results in a liver inflammation score of 0 or 1 (e.g., as described herein).

In some embodiments, the liver of the patient is characterized by hepatocellular ballooning.

In some embodiments, administering of the MAdCAM-1 antibody results in a decrease in hepatocellular ballooning.

In some embodiments, administering of the MAdCAM-1 antibody results in ballooning score of 0.

In some embodiments, treatment commences independently of determining a NAS score.

In some embodiments, methods and uses described herein result in an at least a two-point improvement in NAS without worsening of fibrosis in a patient in need thereof.

In some embodiments, a patient has liver fibrosis.

In some embodiments, fibrosis scored/staged with values of 0-4 (Table 6, below).

TABLE 6
Fibrosis Scoring/Staging

| Stage | Score | Description |
|---|---|---|
| 0 | 0 | No fibrosis |
| 1* | 1A | mild, zone 3, perisinusoidal fibrosis |
| | 1B | moderate zone 3, perisinusoidal fibrosis |
| | 1C | portal/periportal fibrosis |
| 2 | 2 | perisinusoidal and portal/periportal |
| 3 | 3 | bridging fibrosis |
| 4 | 4 | cirrhosis |

*perisinusoidal or periportal fibrosis

In some embodiments, a patient has non-cirrhotic NASH.

In some embodiments, a patient has cirrhotic NASH.

In some embodiments, a patient has compensated cirrhotic NASH.

In some embodiments, a patient has a fibrosis stage score of 0-3. In embodiments, a patient has a fibrosis stage score of 0. In some embodiments, a patient has a fibrosis stage score of 1. In some embodiments, a patient has a fibrosis stage score of 2. In embodiments, a patient has a fibrosis stage score of 3. In some embodiments, a patient has a fibrosis stage score of 4 (cirrhosis). In some embodiments, after-treatment patients can have a fibrosis stage score that is at least no worse than the baseline score before treatment, and alternatively can have a reduction in the fibrosis stage score of at least one level, alternatively at least two or three levels.

In some embodiments, methods and uses described herein result in no increase in the patient's liver fibrosis score (stabilization of liver fibrosis in the patient).

In some embodiments, methods and uses described herein result in a decrease of at least one (≥1) in the patient's liver fibrosis score (reversal of liver fibrosis in the patient).

In some embodiments, liver fibrosis is characterized using an enhanced liver fibrosis test (ELF) score. In some embodiments, liver fibrosis is characterized as an ELF score of less than 7.7. In some embodiments, liver fibrosis is characterized by an ELF score of greater than or equal to 7.7 and less than 9.8. In some embodiments, liver fibrosis is characterized by an ELF score of greater than 9.8.

In some embodiments, methods and uses described herein result in maintenance (no worsening) of fibrosis stage in a patient in need thereof.

In some embodiments, methods and uses described herein result in fibrosis regression in a patient in need thereof.

In some embodiments, methods and uses described herein result in a decrease in liver hypertrophy in the patient.

In some embodiments, methods and uses described herein result in a decrease in a patient's liver collagen levels.

In some embodiments, methods and uses described herein result in a decrease in a patient's hepatic tissue alpha smooth muscle actin (α-SMA) levels.

In some embodiments, methods and uses described herein result in a decrease in a patient's hepatocyte apoptosis levels.

In some embodiments, methods and uses described herein result in a decrease in a patient's hyaluronic acid levels.

In some embodiments, methods and uses described herein result in a decrease in a patient's tissue inhibitors of metalloproteinases (TIMP-1) levels.

In some embodiments, methods and uses described herein result in a decrease in a patient's procollagen type III terminal peptide (PIIINP) levels.

In some embodiments, methods and uses described herein result in a decrease in a patient's soluble Fas ligand levels.

In some embodiments, methods and uses described herein result in a decrease in a patient's leptin levels.

In some embodiments, methods and uses described herein result in a decrease in a patient's aspartate aminotransferase (AST) to platelet index (APRI).

In some embodiments, methods and uses described herein result in a decrease in a patient's fibrosis 4 (FIB-4) score.

In some embodiments, methods and uses described herein result in a decrease in a patient's liver stiffness.

In some embodiments, methods and uses described herein result in an increase in a patient's adiponectin levels.

In some embodiments, the methods and uses described herein result in a decrease in a patient's neoepitope-specific N-terminal pro-peptide of type III collagen (Pro-C3) levels.

In some embodiments, the methods and uses described herein result in a decrease in a patient's soluble MAdCAM-1 levels.

In some embodiments, the methods and uses described herein result in a decrease in a patient's high-sensitivity C-reactive protein (hsCRP) levels.

In some embodiments, the methods and uses described herein result in a decrease in a patient's calprotectin levels.

In some embodiments, the methods and uses described herein result in a decrease in a patient's Interleukin 8 (IL-8) levels.

### Serum Markers and Biomarkers

In some embodiments, any of the methods and uses described herein result in changes of serum markers (e.g., serum markers of liver pathologies such as liver fibrosis or NASH) in a patient in need thereof. Accordingly, in embodiments, a patient is selected based on a particular expression level of a serum marker (including any described herein). For example, methods described herein can be particularly beneficial to a patient having a particular (e.g., a threshold level) of a serum marker (e.g., any described herein). Further, methods described herein can result in favorable changes in a serum marker (e.g., modulate the level of any serum marker described herein). In some embodiments, methods described herein can result in decreases of elevated serum markers (e.g., any described herein).

For example, non-invasive measures of fibrosis to monitor treatment efficacy can be useful for avoiding the need for repeated liver biopsy (e.g., to identify patients that can benefit from methods described herein, including those having any liver pathology described herein such as NASH). In some embodiments, a serum marker is FIB-4. In some embodiments, a serum marker is APRI. APRI and FIB-4 scores are calculated by the following published formulas (Kim et al., “Association between noninvasive fibrosis markers and mortality among adults with nonalcoholic fatty liver disease in the United States”, Hepatology, 57: 1357 (2013)), wherein “PLT count” is the platelet count; “AST” is aspartate transaminase, and the upper limit of normal is 40 IU/mL; and “ALT” is alanine aminotransferase:

APRI=([AST/upper limit of normal]/PLT count[109/L])

FIB-4=(age[years]×AST[IU/L])/(PLT[109/L]×(ALT[IU/L])1/2).

Accordingly, exemplary serum markers include enzymes such as alanine aminotransferase (ALT), aspartate aminotransferase (AST), alkaline phosphatase (ALP), or γ-glutamyl transferase (GGT), or any combination thereof. In embodiments, a patient has at least one elevated liver enzyme.

Still other exemplary serum markers include: total cholesterol, high-density lipoprotein (HDL)-cholesterol, triglycerides, bilirubin, albumin, C-peptide, apolipoprotein A1, apolipoprotein B, leptin, adiponectin, free fatty acids, ghrelin and tumor necrosis factor-alpha (TNF-α).

In some embodiments, a patient has elevated hepatic alanine aminotransferase (ALT) levels. In some embodiments, methods and uses described herein result in decreased hepatic alanine aminotransferase (ALT) levels. In some embodiments, a patient has ALT levels that fall within normal levels (e.g., about 10-40 IU/L). In some embodiments, a patient has ALT levels that are greater than about 40 IU/L. In some embodiments, ALT is no greater than about 30 IU/L (e.g., for a male patient). In some embodiments, ALT is greater than about 30 IU/L (e.g., for a male patient). In some embodiments, ALT is no greater than about 19 IU/L (e.g., for a female patient). In some embodiments ALT is greater than about 19 IU/L (e.g., for a female patient).

In some embodiments, a patient has elevated hepatic aspartate aminotransferase (AST) levels. In some embodiments, a patient has AST levels that fall within normal levels (e.g., about 10-35 IU/L). In some embodiments, a patient has AST levels that are greater than about 30 IU/L. In some embodiments, a patient has AST levels that are greater than about 35 IU/L. In embodiments, methods and uses described herein result in decreased hepatic aspartate aminotransferase (AST) levels.

In some embodiments, the ratio of aspartate aminotransferase (AST) and alanine aminotransferase (AST) is determined. In some embodiments, a patient has a ratio of AST/ALT that is greater than 1. In some embodiments, a patient has a ratio of AST/ALT that is less than 1.

In some embodiments, methods and uses described herein result in a decrease in a patient's aspartate aminotransferase (AST) to alanine aminotransferase (ALT) ratio.

In some embodiments, methods and uses described herein result in a change in a patient's ratio of aspartate aminotransferase (AST) and alanine aminotransferase (ALT) ratio such that the ratio is closer to 1.

In some embodiments, a patient has elevated alkaline phosphatase (ALP) levels. In some embodiments, a patient has ALP levels that fall within a normal range (e.g., about 20-140 or about 37-116 IU/L). In some embodiments, a patient has ALP levels that are greater than about 120 IU/L or about 140 IU/L. In some embodiments, a patient has ALP levels that are greater than about 150 IU/L. In some embodiments, methods and uses described herein result in decreased alkaline phosphatase (ALP) levels.

In some embodiments, a patient has elevated γ-glutamyl transferase (GGT) levels. In some embodiments, a patient has GGT levels that fall within a normal range (e.g., about 5-30 IU/L or about 9-48 IU/L). In some embodiments, a patient has GGT In some embodiments, a patient has elevated GGT levels that are at least about 50 IU/L. In some embodiments, GGT levels that are up to about 90 IU/L or about 100 IU/L. In some embodiments, methods and uses described herein result in decreased GGT levels.

In some embodiments, a patient has elevated triglyceride levels. In some embodiments, methods and uses described herein result in decreased triglyceride levels.

In some embodiments, a patient has elevated non-esterified fatty acid (NEFA) levels. In some embodiments, methods and uses described herein result in decreased non-esterified fatty acid (NEFA) levels.

In some embodiments, a patient has elevated cholesterol levels. In some embodiments, methods and uses described herein result in decreased cholesterol levels.

In some embodiments, a patient has depressed levels of HDL-cholesterol.

In some embodiments, a patient is selected based on Child-Pugh score. A Child-Pugh score is determined based on total bilirubin (TBL), serum albumin, internal normalization ratio (INR), degree of ascites, and degree of hepatic encephalopathy.

In some embodiments, a patient is selected based on Multiparametric Magnetic Resonance Imaging: Iron-corrected T1.

In some embodiments, a patient is selected based on Model for End stage Liver Disease (MELD) Score. A MELD Score is determined based on serum bilirubin, serum creatinine, and the INR for prothrombin. In some embodiments, the MELD score is <12. In some embodiments, the MELD score is ≥12. In some embodiments, the MELD score is ≥15

In some embodiments, a patient is selected based on West Haven Criteria (WHC) for hepatic encephalopathy.

In some embodiments the surrogate markers are selected from alanine aminotransferase (ALT); aspartate aminotransferase (AST); Controlled Attenuation Parameter (CAP); Enhanced Liver Fibrosis Test (ELF); fibrosis stage (F); FibroScan-aspartate aminotransferase (FAST); Fibrosis-4 Index (FIB-4); formalin-fixed paraffin-embedded (FFPE); high-sensitivity C reactive protein (hsCRP); interleukin (IL); liver stiffness measurement (LSM); and neoepitope specific N-terminal pro-peptide of type III collagen (Pro C3).

In some embodiments, markers are selected from Pro-C, ELF, soluble MAdCAM-1, hsCRP, calprotectin, IL-8, and blood T cell phenotyping.

In some embodiments, surrogate markers (e.g., biomarkers) are selected from markers of circulatory cell populations. In some embodiments, the markers includes those of Th17/Th1 cells. In some embodiments, the markers includes those of Th2 cells. In some embodiments, markers include those of β7⁺ T-cells. In some embodiments, the markers include CCR9. In some embodiments, the markers include CXCR3.

In some embodiments, liver cirrhosis is characterized using an Agile 4 Score. An Agile 4 Score is derived score considers LSM, AST, ALT, platelets, diabetes status and gender.

### Liver Inflammation

In one aspect, the invention features a method for treating (e.g., reducing) liver inflammation, the method comprising administering to the patient in need thereof an effective amount of a MAdCAM-1 antibody.

In some embodiments, liver inflammation is lobular inflammation.

### Hepatocellular Ballooning

In one aspect, the invention features a method for treating (e.g., reducing) hepatocellular ballooning, the method comprising administering to the patient in need thereof an effective amount of a MAdCAM-1 antibody.

Hepatocyte ballooning is a type of cell death visually characterized by hypertrophy and localization of cellular nuclei at or near the center of the cell.

In some embodiments, administering of the MAdCAM-1 antibody results in a ballooning score of 0.

In some embodiments, a patient has non-alcoholic steatohepatitis (NASH).

### Liver Fibrosis (Including Cirrhosis)

In one aspect, the invention features a method for treating liver fibrosis, the method comprising administering to the patient in need thereof an effective amount of a MAdCAM-1 antibody.

In some embodiments, a patient has stage 2, stage 3, or stage 4 liver fibrosis.

In some embodiments, a patient has cirrhosis (stage 4 liver fibrosis).

In some embodiments, a patient has a fibrosis stage score of ≥1.

In some embodiments, a patient has a fibrosis stage score of 0-3.

In some embodiments, a patient has a fibrosis stage score of 0. In embodiments, a patient has a fibrosis stage score of 1. In embodiments, a patient has a fibrosis stage score of 2. In embodiments, a patient has a fibrosis stage score of 3. In embodiments, a patient has a fibrosis stage score of 4 (cirrhosis).

In some embodiments, after treatment patients can have a fibrosis stage score that is at least no worse than the baseline score before treatment, and alternatively can have a reduction in the fibrosis stage score of at least one level, alternatively at least two or three levels.

### Steatosis

In one aspect, the invention features a method for treating steatosis, the method comprising administering to the patient in need thereof an effective amount of a MAdCAM-1 antibody.

In some embodiments, administering of the MAdCAM-1 antibody results in a decrease of steatosis in the patient.

In some embodiments, steatosis comprises macrovesicular steatosis. In embodiments, steatosis comprises microvesicular steatosis. In embodiments, steatosis comprises macrovesicular and microvesicular steatosis.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

A clinical study is provided herein that is conducted to explore the mechanism of action and to evaluate the safety and tolerance of the MAdCAM-1 antibody in participants with nonalcoholic steatohepatitis (NASH).

The clinical study is designed to explore the role of MAdCAM-1 in NASH by administering a MAdCAM-1 inhibitor (i.e., the MAdCAM-1 antibody) and to evaluate the safety and tolerability of 75 mg dose of the MAdCAM-1 antibody in participants with NASH with fibrosis stages 2 through 4 (nonalcoholic fatty liver disease activity score (NAS)≥4 with at least 1 point each in steatosis, ballooning, and lobular inflammation) with different fibrosis stages (fibrosis stage 2-3 and fibrosis stage 4/cirrhotic participants without decompensation). The role of MAdCAM-1 is evaluated by the changes in inflammatory and fibrosis biomarkers and in liver function tests compared with baseline. Multiparametric MRI: cT1 is used to evaluate the fibroinflammatory changes in the liver.

### Example 1: First Study Design and Methodology

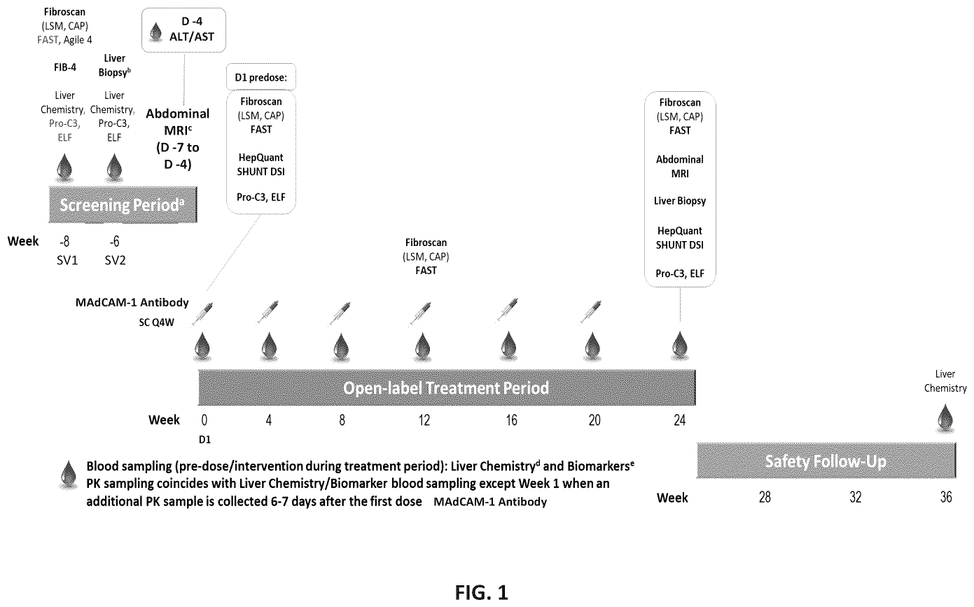

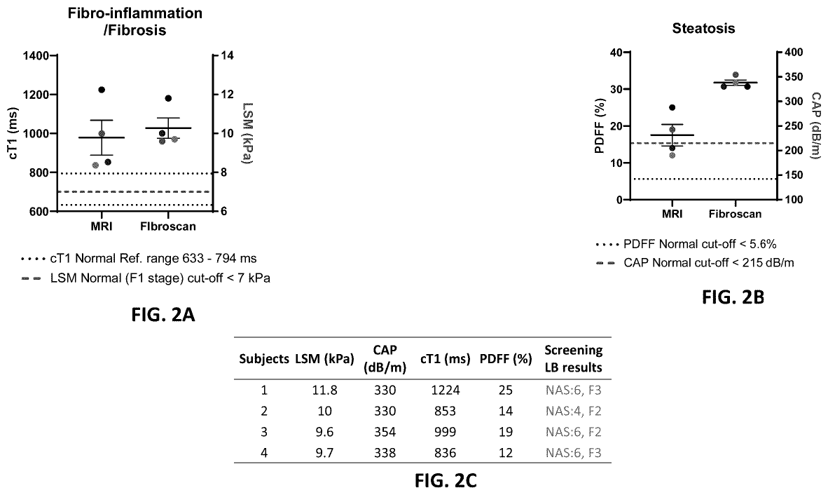

The clinical study is a multicenter, single-arm, open-label, Phase 1b study. A schematic representation of the study design is shown in FIG. 1. The clinical study includes three different periods or phases—screening period, treatment period, and safety follow-up period. The screening period spans over 8 weeks prior to treatment period. The treatment period lasts for 24 weeks that commences immediately after the screening period, and the follow-up period starts immediately after the treatment period and lasts for 12 weeks. Example 2, provided below, lists and describes schedule of all the activities that are performed during each period.

In the study, approximately 30 participants are enrolled to ensure that at least 18 participants (approximately 12 participants with F2/F3 and approximately 6 participants with F4cc) complete the 24-week treatment period. Enrollment of F2 and F3 participants in a 1:1 ratio is not be required.

Participants are aged ≥18 and ≤70 years and are diagnosed with NASH without decompensated cirrhosis or neoplasia. The broad NASH population is targeted, and participants with documented stable type 2 diabetes mellitus (T2DM) or participants with high body mass index>25 kg/m² with ≥1 criterion of metabolic syndrome is permitted to participate in the study.

In the study, screening liver biopsy eligibility is defined by assessments performed at the first screening visit (Week −8), which include a noninvasive test (i.e., FAST score) that incorporates circulating biomarkers (e.g., AST), clinical information (e.g., age) imaging data (e.g., liver stiffness measurement (LSM) by FibroScan and liver fat content by Controlled Attenuation Parameter (CAP), as well as serum Pro-C3 (neoepitope specific N-terminal propeptide of type III collagen) levels and the Enhanced Liver Fibrosis (ELF) score (consisting of tissue inhibitor of metalloproteinases 1 (TIMP-1), amino-terminal propeptide of type III procollagen (PIIINP) and hyaluronic acid (HA). Participants with FAST score>0.35, Pro-C3≥12.6 ng/ml and ELF score≥7.7 (in addition to other inclusion and exclusion criteria, such as ALT and AST levels) at Week −8 are eligible for liver biopsy that is performed at the Week −6 visit during screening. Evidence for NASH (NAS≥4 with at least 1 point each in steatosis, ballooning, and lobular inflammation) and liver fibrosis stage F2 through F4cc in the liver biopsy enable participants to enroll in the study.

Historical liver biopsy samples are acceptable for screening purposes if collected between 24 weeks and 4 weeks before the start of the screening period. For any F4 participants who meet the inclusion and exclusion criteria before establishment of safety and tolerability in the first four F2/F3 participants, historical liver biopsy samples are acceptable for screening purposes if collected within 10 weeks before the screening period (maximum allowed time between historical biopsy and first dose of study drug is 34 weeks for any participant). The first screening visit must occur at least 4 weeks after the historical biopsy to ensure that any biopsy-induced changes in liver function parameters or biomarkers have stabilized. If a qualifying historical liver biopsy sample is available to be reviewed and read centrally by the study pathologists and if it meets all the criteria as defined in the histopathology manual, liver biopsy need not be repeated during screening. Historical liver biopsy samples must show evidence for NASH (NAS≥4 with at least 1 point each in steatosis, ballooning, and lobular inflammation) and liver fibrosis stage F2 through 44cc. Participants with qualifying historical liver biopsy must have adhered to restrictions on prohibited hepatotoxic medications during the specified period before historical liver biopsy sampling. For participants with qualifying historical liver biopsies, the baseline abdominal MRI visit may occur as soon as possible after liver chemistry results from Screening Visit 2 become available, and Screening Visit 2 and the Day −4 visit (Visit 4a2) must be at least 2 weeks apart.

During the screening period Pro-C3, ELF, and other exploratory markers are measured at 3 visits (including Day 1 pre-dose) to determine intra-participant variability. This enables the correct interpretation of the post-treatment decrease in these biomarkers relative to the intra-participant variability at baseline.

After safety and tolerability is established in the first four F2/F3 participants who receive at least 3 doses of MAdCAM-1 antibody 75 mg and have been monitored for at least 12 weeks after the first dose, eligible F4cc participants will enroll into the study. The screening period may be extended for up to 24 weeks for participants with results consistent with F4 fibrosis (i.e., compensated cirrhosis) after the first screening visit (i.e., a combination of FIB-4≥3.48 and LSM≥20 kPa, or an Agile 4 score≥0.57), or have a screening liver biopsy or a historical liver biopsy that confirms F4 NASH (NAS≥4 with at least 1 point each in steatosis, ballooning, and lobular inflammation), until safety and tolerability data are evaluated in the first four F2/F3 participants.

All eligible participants (F2-F4) will enter a 24-week, single-arm, open-label treatment period with anti-MAdCAM-1 antibody 75 mg. Anti-MAdCAM-1 antibody will be administered subcutaneously (SC) Q4W for 24 weeks (last dose at Week 20), followed by a 12-week safety follow-up period. Liver biopsy samples will be collected during screening and at Week 24. Fibro-inflammation will be assessed by cT1 MRI, liver stiffness by FibroScan, and hepatocellular function by HepQuant-SHUNT DSI (optional if the site is unable to perform this assessment but not optional for the participant if the site has the capability to perform this assessment) at the start of the treatment period and at Week 24. At visits Q4W between baseline and Week 24, assessments of selected biomarkers (including but not limited to Pro-C3, ELF, soluble MAdCAM-1, hsCRP, and calprotectin), liver chemistry tests, and specific safety data collection (laboratory, adverse events [AEs], neurological assessments) will be performed. Liver stiffness measurement by FibroScan will also be performed at Week 12. Safety and tolerability data will be continuously monitored and will be evaluated by an internal group independent from the study team. All participants, including those who discontinue, undergo a safety follow-up period through 12 weeks after the last dose of study drug. The end of the study is defined as the date of the last visit (Week 36) of the last participant undergoing the study.

The participant's maximum duration of participation in the study is expected to be approximately 46 weeks (11.5 months), with the exception of any F4 cirrhotic participants who have met the inclusion and exclusion criteria (provided herewith) before safety and tolerability have been established in four F2/F3 participants. For these participants, the total study period is expected to be approximately 60 weeks (15 months).

### Example 2: Schedule of Study Activities

Numerous activities are performed during screening period, treatment period, follow-up and end of study period, and are listed below under the schedule of activities.

### Schedule of Study Activities—Screening Period

Table 7 provides lists of activities performed and/or checked during the screening period of the study.

TABLE 7
Schedule of Study Activities: Screening Period

| Visit | 1 | 2 |
|---|---|---|
| Week | −8 | −6 |
| Day | −56 | −42 |
| Visit Window (Days) | NA | +14 |
| On-site Visit | Yes | Yes |

Administrative Procedures

| | | |
|---|---|---|
| Informed consent | X | |
| Inclusion/exclusion criteria | X | X |
| Demographics and medical history | X | |
| Concurrent medical conditions | X | |
| Medication and procedure history | X | |
| Concomitant medication review | X | Throughout the |
| | | study from the |
| | | signing of |
| | | informed consent |
| | | form onwards |
| Pregnancy avoidance counseling | X | |

Clinical Procedures

| | | |
|---|---|---|
| AE assessment | X | Throughout the |
| | | study from the |
| | | signing of |
| | | informed consent |
| | | form onwards |
| Height | X | |
| Body weight | X | X |
| Vital signs | X | X |
| Complete physical examination | X | X |
| Targeted neurological assessment | X | X |
| 12-lead ECG | X | |
| Chest X-ray | X | |
| Abdominal ultrasound | X | |

Clinical Laboratory Assessments

| | | |
|---|---|---|
| Blood sample-clinical chemistry panel | X | X |
| Blood sample-hematology panel | X | |
| Coagulation (INR) | X | X |
| Serology | X | |
| Infectious disease panel | X | |
| HbA1c | X | |
| eGFR (CKD-EPI) | X | |
| Creatine kinase | X | |
| Vitamin D | X | |
| AMA | X | |
| ANA, SMA, alpha-1-antitrypsin, and | X | |
| ceruloplasmin | | |
| IgG4 | X | |
| TB test (PPD or IGRA) | X | |
| Serum α-fetoprotein | X | |
| FSH^{q} | X | |
| Serum β-hCG^{r} | X | |
| Urine β-hCG^{r} | | X |
| Urinalysis | X | X |
| Urine drug screening | X | X |
| Alcohol screening (AUDIT) | X | |
| FIB-4 score | X | |
| FAST score | X | |
| Agile 4 score | X | |
| Child-Pugh score | X | |
| MELD score | X | |

Clinical Procedures

| | | |
|---|---|---|
| LSM, CAP (FibroScan) | X | |
| Liver biopsy^{t} | | X |

Biomarkers

| | | |
|---|---|---|
| Serum sample for Pro-C3 | X | X |
| Serum sample for ELF score | X | X |
| Serum sample for soluble MAdCAM-1 | X | X |
| Serum sample for hsCRP | X | X |
| Plasma sample for calprotectin | X | X |
| Serum sample for circulating biomarkers | X | X |

AE = adverse event;
AMA = antimitochondrial antibody;
ANA = antinuclear antibody;
AUDIT = Alcohol Use Disorders Identification Test;
β-hCG = beta-human chorionic gonadotropin;
CAP = Controlled Attenuation Parameter;
CKD-EPI = Chronic Kidney Disease Epidemiology Collaboration;
ECG = electrocardiogram;
eGFR = estimated glomerular filtration rate;
ELF = Enhanced Liver Fibrosis;
FAST = FibroScan-aspartate aminotransferase;
FIB-4 = Fibrosis-4 Index;
FSH = follicle-stimulating hormone;
HbA1c = hemoglobin A1c;
HBcAb = hepatitis B core antibody;
HBsAg = hepatitis B surface antigen;
HBV = hepatitis B virus;
HCVAb = hepatitis C virus antibody;
HIV = human immunodeficiency virus;
hsCRP = high-sensitivity C-reactive protein;
IGRA = interferon-gamma release assay;
IgG4 = immunoglobulin G4;
INR = international normalized ratio;
LSM = liver stiffness measurement;
MAdCAM-1 = mucosal addressin cell adhesion molecule-1;
MELD = Model for Endstage Liver Disease;
NA = not applicable;
PML = progressive multifocal leukoencephalopathy;
PPD = purified protein derivative;
Pro-C3 = neoepitope-specific N-terminal propeptide of type III collagen;
SAE = serious adverse event;
SMA = smooth muscle antibody;
TB = tuberculosis

The term “Informed consent” indicates that the informed consent process must be completed, signed and dated before proceeding.

The term “Pregnancy avoidance counseling” relates to lifestyle restrictions for example, contraception and breastfeeding. The term “AE assessment” indicates adverse events must be collected from the time participant signs the informed consent form. AE/SAE review.

The term “Vital signs” indicates vital signs include that includes blood pressure (resting more than 5 minutes), pulse, respiratory rate, and temperature.

The term “Targeted neurological assessment” indicates that the Participants are evaluated to reveal any potential abnormalities in the following neurological domains: vision, motor, tactile sensation, coordination/cerebellar function, speech, verbal comprehension, and cognition/behavior. Participants with any unexplained positive item during screening that is suggestive of progressive multifocal leukoencephalopathy (PML) are excluded.

The term “Chest x-ray” indicates that chest x-rays performed up to 12 weeks before the first screening visit is optionally used if available

“Blood sample-clinical chemistry panel” or “blood sample-hematology panel” indicate laboratory tests such as blood glucose and triglycerides are collected in a fasting state.

“Serology panel” indicates that the panel includes HIV (HIV-Ab/Ag), hepatitis B surface antigen (HBsAg), heptatitis B core antibody (HBcAb), and hepatitis C (HCVAb). Participants who test negative for HBsAg but positive for HBcAb would be considered eligible if HBV DNA results are negative (as confirmed by HBV DNA PCR reflex testing). Participants with positive HBV DNA test results are not be eligible. Participants who are HCVAb positive without evidence of HCV RNA may be considered eligible (spontaneous viral clearance or previously treated and cured [defined as no evidence of HCV RNA at least 12 weeks before baseline]).

“Infection Disease Panel” indicates that the infection panel includes cytomegalovirus, Epstein-Barr virus, and herpes simplex virus.

IGRA screening may be repeated during the screening period if the initial result is indeterminate. The participant may be enrolled if confirmatory IGRA results from the central laboratory are negative. If the confirmatory IGRA results are positive, the participant should be screen-failed. If both initial and confirmatory IGRA results are indeterminate, the participant may be enrolled after a Mantoux tuberculin skin test is negative and after consultation with the sponsor and a pulmonary or infectious disease specialist who determines low risk of infection and confirms no risk of immunosuppressive treatment. If the time for consultation exceeds the visit window, the participant should be screen-failed and can be rescreened after the consultation establishes eligibility for study participation. This consultation must be included in the participant's medical history.

“FSH” indicates that the follicle stimulating hormone is assessed for postmenopausal participants.

“Serum β-hCG” indicates that pregnancy tests only apply to participants of childbearing potential. A confirmatory serum β-hCG pregnancy test is only required for participants who have a positive urine pregnancy test at any time.

“Liver biopsy” indicates that the eligibility for the liver biopsy at Week −6 is determined by data generated from Week −8 assessments: FAST score≥0.35, Pro-C3 levels≥12.6 ng/mL, and ELF score≥7.7 in addition to other inclusion and exclusion criteria at Week −8. Liver chemistry-related samples at Week −8 must be evaluated and eligibility determined before the screening liver biopsy can be performed. For participants who meet other eligibility criteria at Week −8, if ALT/AST results at Week −8 are >4×ULN and <5×ULN, then the ALT/AST results at Week −6 will also be evaluated before liver biopsy and abdominal MRI can be performed.

“Circulating Biomarkers” indicates repeat samples collected at the same time of day across screening.

“Other exploratory blood biomarker sampling” indicates that the exploratory biomarkers include (but are not limited to) markers of circulatory cell populations such as but not limited to Th17/Th1 vs. Th2, β7+ T cells, CCR9 and CXCR3.

### Schedule of Activities—Treatment Period, Follow-Up, and End of Study

Table 8 provides lists of activities performed during the treatment period, follow-up and end of study period.

TABLE 8
Schedule of Activities: Treatment Period, Follow-up, and End of Study

| Period | | | | | | | | | | | |
|---|---|---|---|---|---|---|---|---|---|---|---|
| Treatment Period | | | | | | | | | Follow-up | | |
| Visit | | | | | | | | | | | |
| | | | | | | | | 11/End of | | | |
| 4a | 4b | | | | | | | Treatment/ | | | |
| Baseline | Baseline | | | | | | | Early | | | 14/End |
| Part 1, 2 | Part 3 | 5 | 6 | 7 | 8 | 9 | 10 | Termination | 12 | 13 | of Study |
| Week | | | | | | | | | | | |
| −1 | 0 | 1 | 4 | 8 | 12 | 16 | 20 | 24 | 28 | 32 | 36 |
| Day | | | | | | | | | | | |
| −7, −4 | 1 | 8 | 29 | 57 | 85 | 113 | 141 | 169 | 197 | 225 | 253 |
| Visit Window (Days) | | | | | | | | | | | |
| ±3, −2 | | ±3 | ±3 | ±3 | ±3 | ±3 | ±3 | ±3 | ±7 | ±7 | ±7 |
| On-site visit | | | | | | | | | | | |
| Yes, Yes | Yes | Yes | Yes | Yes | Yes | Yes | Yes | Yes | Yes | Yes | Yes |

Administrative Procedures

| | |
|---|---|
| Concomitant medication review | Throughout the study from the signing of informed consent form onwards |

Clinical Procedures

| AE assessment | Throughout the study from the signing of informed consent form onwards | | | | | | | | | | |
|---|---|---|---|---|---|---|---|---|---|---|---|
| Body weight | | X | X | X | X | X | X | X | X | X | X |
| Vital signs | | X | X | X | X | X | X | X | X | X | X |
| Complete physical examination | | X | X | X | X | X | X | X | X | X | X |
| Targeted neurological assessment | | X | X | X | X | X | X | X | X | X | X |
| 12-lead ECG | | X | | | | | | X | | | |

Clinical Laboratory Assessments

| | | | | | | | | | | | |
|---|---|---|---|---|---|---|---|---|---|---|---|
| Blood sample-clinical chemistry panel | X | X | X | X | X | X | X | X | | | X |
| Blood sample-hematology panel | X | X | X | X | X | X | X | X | | | X |
| Coagulation (INR) | X | | | | | | | X | | | X |
| HbA1c | X | | | | X | | | X | | | X |
| eGFR (CKD-EPI) | X | | | | X | | | X | | | X |
| Creatine kinase | X | | | | X | | | X | | | X |
| Urine β-hCG | X | | X | X | X | X | X | X | X | X | X |
| Urinalysis | X | | X | X | X | X | X | X | X | X | X |
| Urine drug screening | X | | X | X | X | X | X | X | | | X |
| Alcohol screening (AUDIT) | X | | | | X | | | | | | X |
| FIB-4 score | X | | | | X | | | X | | | |
| FAST score | X | | | | X | | | X | | | |
| Child-Pugh score | X | | | | | | | X | | | |
| MELD score | X | | | | | | | X | | | |

Clinical Procedures

| | | | | |
|---|---|---|---|---|
| LSM, CAP (FibroScan ™) | | X | X | X |
| Liver biopsy | | | | X |
| Abdominal MRI (for liver cT1 and other | X | | | X |
| MRI-based exploratory biomarkers) | | | | |
| HepQuant-SHUNT DSI | | X | | X |

PK & Biomarkers

| | | | | | | | | | | | |
|---|---|---|---|---|---|---|---|---|---|---|---|
| Serum sample for PK | X | X | X | X | X | X | X | X | | | |
| Serum sample for immunogenicity | X | | | | X | | | X | X | X | X |
| Serum sample for Pro-C3 | X | X | X | X | X | X | X | X | | | X |
| Serum sample for ELF score | X | X | X | X | X | X | X | X | | | X |
| Serum sample for soluble MAdCAM-1 | X | X | X | X | X | X | X | X | | | X |
| Serum sample for hsCRP | X | | X | X | X | X | X | X | | | X |
| Plasma sample for calprotectin | X | | X | X | X | X | X | X | | | X |
| Serum sample for circulating biomarkers | X | | X | X | X | X | X | X | | | X |
| Blood sample for flow cytometry | X | | | | X | | | X | | | |
| Whole blood transcriptomics | X | | | | X | | | X | | | |

Treatment Procedures

| | | | | | | | | | | |
|---|---|---|---|---|---|---|---|---|---|---|
| MAdCAM-1 antibody administration | X | X | X | X | X | X | | | | |
| Hypersensitivity monitoring | X | X | X | X | X | X | X | X | X | X |

AE = adverse event;
AUDIT = Alcohol Use Disorders Identification Test;
β-hCG = beta-human chorionic gonadotropin;
CAP = Controlled Attenuation Parameter;
CKD-EPI = Chronic Kidney Disease Epidemiology Collaboration;
cT1 = iron-corrected T1 mapping;
DSI = Disease Severity Index;
ECG = electrocardiogram;
eGFR = estimated glomerular filtration rate;
ELF = Enhanced Liver Fibrosis;
FAST = FibroScan-aspartate aminotransferase;
FIB-4 = Fibrosis-4 Index;
HbA1c = hemoglobin A1c;
hsCRP = high-sensitivity C-reactive protein;
Ig = immunoglobulin;
INR = international normalized ratio;
LSM = liver stiffness measurement;
MAdCAM-1 = mucosal addressin cell adhesion molecule-1;
MELD = Model for Endstage Liver Disease;
MRI = magnetic resonance imaging;
PK = pharmacokinetics;
Pro-C3 = neoepitope-specific N-terminal propeptide of type III collagen;
SAE = serious adverse event

“AE assessment” indicates that all adverse events are collected from the time participant signs the informed consent form.

“Vital signs” include blood pressure (resting more than 5 minutes), pulse, respiratory rate, and temperature.

“Targeted neurological assessment” indicates that the participants are

evaluated to reveal any potential abnormalities in the following neurological domains: vision, motor, tactile sensation, coordination/cerebellar function, speech, verbal comprehension, and cognition/behavior.

“Laboratory tests” indicates that blood glucose and triglycerides are collected in a fasting state.

Liver biopsy must be conducted as the last assessment for the Week 24/End of Treatment/Early Termination visit.

“Urine β-hCG indicates” that pregnancy tests only apply to participants of childbearing potential. A confirmatory serum β-hCG pregnancy test is only required for participants who have a positive urine pregnancy test at any time.

“Whole blood T-cell Phenotyping” indicates that exploratory biomarkers include (but are not limited to) markers of circulatory cell populations such as but not limited to Th17/Th1 vs. Th2, β7⁺ T cells, CCR9 and CXCR3.

“MAdCAM-1 antibody administration” indicates study drug administration.

Hypersensitivity monitoring indicates that the participants are monitored for the presence of Type I (anaphylaxis) and Type III (immune complex) hypersensitivity reactions. Participants who experience AEs suggestive of Type I and Type III hypersensitivity reactions have blood samples (3 mL and 4 mL, respectively) collected for anti-MAdCAM IgE antibody determination (Type I) and C3a and C5b 9 (Type III) determination, respectively.

### Example 3: Study Population

Study population, i.e., participants, is governed by the exclusion and inclusion criteria provided herewith.

### Inclusion Criteria

All participants must meet all of the following criteria to be eligible for inclusion in the study:

The participant is aged 18 to 70 years, inclusive, at the time of signing the ICF.

The participant has FAST≥0.35 (applies to participants without qualifying historical liver biopsy at enrollment). FAST score of >0.35 at enrollment is not required for participants with a qualifying historical liver biopsy that provides evidence of liver fibrosis stage F2, F3, or F4cc according to NASH CRN).

The participant has signs of fibrogenic activity (Pro-C3>12.6 ng/mL, ELF score≥7.7) at the Week −8 screening visit (applies to all participants regardless of whether historical biopsy results are available at screening).

The participant has a diagnosis of NASH confirmed via biopsy (NAS score≥4 with at least 1 point each in steatosis, ballooning, and lobular inflammation) and liver fibrosis stage F2, F3, or F4cc according to NASH CRN, and established according to appropriate guidelines.

The participant is a male participant or a nonpregnant, nonlactating female participant who, if sexually active, agrees to comply with the contraceptive requirements of the protocol, or a female participant of nonchildbearing potential. Participants of reproductive potential who are sexually active must agree to use appropriate contraception (i.e., highly effective methods for female participants and medically appropriate methods for male participants) for the duration of the study and for at least 12 weeks after the last dose of study drug.

The participant, if capable of breastfeeding, agrees to forego breastfeeding for the period from informed consent until 12 weeks after the last dose of study drug.

### Exclusion Criteria

The participants are excluded from the study if any of the following exclusion criteria are met:

### Medical Conditions:

The following laboratory findings are exclusionary for all participants if found during either screening visit (Week −8 or −6) or at Visit 4a2 (Day −4):
a. AST levels>5×the upper limit of normal (ULN).
b. ALT levels>5×ULN

The following laboratory findings are exclusionary for all participants if found during screening (Week −8 or Week −6):
a. ALP≥2×ULN.
b. Serum creatinine≥1.5×ULN, or has an estimated glomerular filtration rate (eGFR)<45 mL/min/1.73 m2.
c. INR≥1.3 (except for participants who are receiving anticoagulant treatment).
d. TBL≥ULN (except for patients with a documented history of Gilbert's syndrome if direct bilirubin is within normal reference range).
e. Direct bilirubin≥3×ULN.
f. Platelet count<60×10⁹/L.

The participant has been diagnosed with decompensated liver disease, or has new signs of decompensation and/or clinically meaningful change in disease status based on the judgment of the investigator (including but not limited to clinically significant changes in TBL, albumin, INR, creatinine, and/or AST and ALT levels) are observed during the screening period, or has any of the following during the screening period:
a. Presence or history of ascites, hepatic encephalopathy, or variceal bleeding.
b. Presence or history of Child-Pugh>6 (Class B or C), unless due to therapeutic anti coagulation.
c. Presence or history of MELD score>12.

The participant has other diagnosed causes of liver disease based on medical history and/or baseline evaluation of laboratory and/or histology results, including, but not limited to viral (e.g., chronic hepatitis B/hepatitis B virus surface antigen [HBsAg] positive, or hepatitis B core antibody [HBcAb] positive; or chronic hepatitis C/hepatitis C virus antibody [HCVAb] positive and hepatitis C virus [HCV] RNA positive; or human immunodeficiency virus [HIV]-antibody positive), alcoholic (alcohol consumption greater than 4 unit son any day or 14 units per week for male participants, or greater than 3 units on any day or 7 units/week for demail participants (1 unit of alcohol is present in one 12 oz/355 mL beer (approximately 5% alcohol), one 5 oz/148 mL glass of wine (approximately 12% alcohol), and one 1.5 oz/44 mL measure of 80-proof liquor (approximately 40% alcohol), or autoimmune conditions, e.g., PSC, primary biliary cirrhosis, autoimmune hepatitis, drug-induced hepatotoxicity, and other rare liver disease, e.g., alpha-1-antitrypsin deficiency, Wilson disease, hemochromatosis.

If a participant tests negative for HBsAg but positive for HBcAb, the participant would be considered eligible if no presence of hepatitis B virus (HBV) DNA is confirmed by HBV DNA polymerase chain reaction (PCR) reflex testing performed by the central laboratory. Participants who are HCVAb positive without evidence of HCV RNA may be considered eligible (spontaneous viral clearance or previously treated and cured [defined as no evidence of HCV RNA at least 12 weeks before baseline]).

The participant has a history of impaired hemostasis that, in the investigator's judgement, would increase the risk to the participant if he or she participates in the study. The participant has a significant concurrent medical condition at the time of screening or baseline, including, but not limited to, the following:
a. Any major illness/condition or evidence of an unstable clinical condition (e.g., hepatic, renal, hematologic, gastrointestinal (GI), endocrine (e.g., unstable diabetes or type 1 diabetes mellitus, or thyroid disease), neurological (pre-existing demyelinating disorder such as multiple sclerosis or new onset seizures, unexplained sensory motor, or cognitive behavioral, neurological deficits, or significant abnormalities noted during screening], cardiovascular, pulmonary, immunologic (e.g., Felty's syndrome), or local active infection/infectious illness (any bacterial, fungal or viral, e.g., clinically active cytomegalovirus, Epstein-Barr virus, herpes simplex virus) that substantially increases the risk to the participant. Participants with hemoglobin A1c (HbA1c)≥6.5% at screening (Week −8) without a previous diagnosis of T2DM must not take part in the study. Participants with a previous diagnosis for T2DM are permitted to enter the study if on a stable regimen of antidiabetic therapy for at least 90 days before screening. Participants who are on a stable regimen of antidiabetic therapy for at least 90 days before screening (Week −8) and have HbA1c of ≥8% at screening (Week −8) should be excluded. Participants with uncontrolled T2DM (i.e., have an HbA1c≥9% should be excluded.
b. Presence of acute coronary syndrome (e.g., acute myocardial infarction, unstable angina pectoris) within 24 weeks before screening.
c. History of significant cerebrovascular disease within 24 weeks before screening.
d. Cancer or history of cancer, including hepatocellular carcinoma or choangiocarcinoma, or lymphoproliferative disease within the previous 5 years (other than resected cutaneous basal cell carcinoma, squamous cell carcinoma, or carcinoma in situ of the uterine cervix that has been treated with no evidence of recurrence).
e. Any other severe acute or chronic medical or psychiatric condition or laboratory or ECG abnormality that may increase the risk associated with study participation or study drug administration or may interfere with the interpretation of study results and, in the judgment of the investigator, would make the participant inappropriate for entry into this study.
f. Transplant organ or history of liver transplantation.
g. Significant trauma or major surgery within 4 weeks before the screening visit, or with any major elective surgery planned to occur during the study.

The participant has severe active inflammatory bowel disease (IBD). Participants with inactive IBD or active of mild to moderate severity that was documented either by prior endoscopy or in previous medical records for >6 months are permitted to enter if they do not require biological treatment or JAK inhibitors.

The participant has a severe immune-mediated inflammatory disease (IMID) (e.g, rheumatoid arthritis, spondylarthrites disease spectrum, connective tissue disorders, cutaneous inflammatory conditions such as psoriasis, atopic dermatitis, hidradenitis suppurativa, asthma, multiple sclerosis). Participants with inactive IMID or active IMID of mild to moderate severity are permitted to enter the study.

The participant has a change in body weight≥5% 3 months before start of the screening or after qualifying liver biopsy. If the participant had a liver biopsy within 6 months of screening, but experienced a weight change of ≥5% since the date of liver biopsy, the liver biopsy must be repeated at screening.

The participant has any laboratory abnormality or condition that, in the investigator's opinion, could adversely affect the safety of the participant or impair the assessment of study results.

The participant has known hypersensitivity to the MAdCAM antibody or formulation excipient.

The participant has active or latent infection with Mycobacterium tuberculosis (TB) who have not completed a generally accepted full course of treatment before screening.

The participant has abnormal chest X-ray findings at screening (Visit 1) or within up to 12 weeks before the first screening if available), such as presence of active TB, general infections, heart failure, or malignancy.

The participant has a positive interferon-gamma release assay (IGRA) at screening or within 12 weeks before screening even in the absence of previously diagnosed active or latent TB. IGRA screening may be repeated during the screening period if the initial result is indeterminate. The participant may be enrolled if confirmatory IGRA results from the central laboratory are negative. If the confirmatory IGRA results from the central laboratory are positive, the participant should be screen-failed. If both initial and confirmatory IGRA results are indeterminate, the participant may be enrolled after a Mantoux tuberculin skin test is negative and after consultation with the sponsor and a pulmonary or infectious disease specialist who determines low risk of infection (ie, the participant would be acceptable for immunosuppressant [eg, anti-tumor necrosis factor (TNF)] treatment without additional action). If the time for consultation exceeds the visit window, the participant should be screen-failed and can be rescreened after the consultation establishes eligibility for study participation. This consultation must be included in the participant's medical history.

The participant has any unexplained symptoms suggestive of PML based on the targeted neurological assessment during the screening period.

The participant has human immunodeficiency virus (HIV).

The participant has active Severe Acute Respiratory Syndrome coronavirus (SARS-COV-2) infection.

The participant has a medical condition (e.g., morbid obesity, claustrophobia) that prevent execution of protocol procedures including percutaneous liver biopsy, LSM by FibroScan, and MRI.

### Prior or Concomitant Therapy:

The participant is receiving treatment with vitamin E, thiazolidinediones (TZDs), or glucagon-like peptide-1 receptor agonists (GLP-1 RA) unless on a stable dose for 3 months before qualifying liver biopsy and not initiated after qualifying liver biopsy and is anticipated to maintain the same dosing regimen throughout study participation

The participant is using drugs, herbs, or supplements historically associated with causing or worsening NAFLD/NASH within 6 months before qualifying liver biopsy or any time after qualifying liver biopsy is performed, including the use of total parenteral nutrition.

The participant has a positive urine screen for amphetamines, cocaine, or opioids at screening.

The participant is receiving methadone or buprenorphine unless on stable maintenance treatment for at least 6 months before screening. Participants with a positive urine drug screen due to prescription opioid-based medication are eligible if the prescription and diagnosis are reviewed and approved by the investigator.

The participant has received a live (attenuated) vaccine within 4 weeks before baseline or is anticipated to receive a live vaccine during the study.

### Excluded Procedures and Treatments:

The participant is using any prohibited concomitant medications as described below. Table 9 details the minimum required time before baseline (Day 1) for common prior treatments that are excluded medications for this study.

TABLE 9
Common Excluded Treatments

| Prohibited Concomitant | | Minimum Required Time | |
|---|---|---|---|
| Medication or | Permanently | Before Baseline (Day 1) | |
| Therapy | Excluded | 30 days | 90 days |
| The MAdCAM-1 antibody in any previous | X | | |
| study | | | |
| Anti-integrin or antiadhesion molecule | | | X |
| treatment (eg, natalizumab, efalizumab, | | | |
| etrolizumab, vedolizumab, or any other | | | |
| investigational anti-integrin/adhesion | | | |
| molecule) | | | |
| Investigational products | | X | |
| Live (attenuated) vaccine | | X | |
| Leukocyte apheresis or selective | | X | |
| lymphocyte, monocyte, or granulocyte | | | |
| apheresis or plasma exchange | | | |
| Biologics with immunomodulatory | | | X |
| properties (such as anti-TNFs) including | | | |
| biosimilars (including ustekinumab) | | | |
| Nonbiologics with immunomodulatory | | | X |
| properties (such as JAK inhibitors) | | | |

In Table 9, TNF stands for tumor necrosis factor. The minimum time for investigational products is 30 days or 5 half-lives if longer. The minimum required time before baseline (Day 1) for non-biologics with immunomodulatory properties is 90 days with the exception of participants who are on stable doses of nonbiologic therapies with immunomodulatory properties (eg, azathioprine, 6-mercaptopurine, or methotrexate).

Antiplatelet and anticoagulant medications must be temporarily discontinued, if safe to do so, before liver biopsies during the study as described below (Neuberger et al., “Guidelines on the use of liver biopsy in clinical practice from the British Society of Gastroenterology, the Royal College of Radiologists and the Royal College of Pathology”, Gut, 69(8), 1382-403 (2020)), or according to local guidelines if more stringent:
Clopidogrel, prasugrel, ticagrelor: stop for 7 days before liver biopsy
Aspirin: stop for 3 to 7 days before liver biopsy
Dual antiplatelet therapy (eg, aspirin/clopidogrel): stop clopidogrel as above but continue aspirin
Dipyridamole: omit on day of liver biopsy
Low molecular weight heparin: stop for 12 hours before liver biopsy for prophylactic dose; stop for 24 hours before liver biopsy for higher than prophylactic dose
Direct oral anticoagulants: stop for 2 days before liver biopsy

Systemic administration of antibiotics that can potentially influence the composition of intestinal flora (including but not limited to clindamycin, ciprofloxacin, amoxicillin/clavulanic acid, cefprozil, imipenem, colistin/amoxicillin, amoxicillin/clavulanate potassium, tetracycline) are prohibited for more than 2 weeks within 3 months before first dose of study drug (and if applicable, within 3 months before a qualifying historical liver biopsy and not initiation after qualifying liver biopsy) and are excluded while on study.

The participants must not take any drug from the list of prohibited drugs within 6 months before the first dose of study intervention and are excluded while on study: Tamoxifen, Amiodarone, alcohol (as described below), Griseofulvin, total parenteral nutrition, obeticholic acid, Valproate, nucleoside analogues (except acyclovir), estrogens at doses greater than 2 mg once daily used for hormone replacement, anabolic steroids, any known hepatotoxins including over-the-counter therapies and herbal therapies such as germander, chaparral, and ma-huang.

Consumption of alcohol greater than 21 units/week for males or 14 units/week for females (1 unit/30 mL of alcohol is present in one 12 oz/360 mL beer, one 4 oz/120 mL glass of wine, and a 1 unit/30 mL measure of 40% proof alcohol) is not permitted within 24 weeks before the first dose of study intervention.

This is not a comprehensive list. Treatments not listed above are generally considered allowable, unless considered a potential hepatotoxin.

### Prior/Concurrent Clinical Study Experience Exclusions

The participant has participated in another investigational study of a drug or device within 1 month before or within 5 half-lives of the prior investigational agent (whichever is longer) before screening.

The participant has participated in another investigational study targeting NASH, T2DM, or obesity within 6 months before screening.

The participant is concurrently participating in another therapeutic clinical study.

The participant has previously received the MAdCAM antibody.

The participant has had previous exposure to anti-integrin or anti-adhesion molecule treatment, e.g., natalizumab, efalizumab, etrolizumab, vedolizumab, or any other investigational anti-integrin/adhesion molecule within 90 days before baseline.

The participant has had previous exposure to any other biologic drugs with immunomodulatory properties such as anti-tumor necrosis factor (anti-TNF), including biosimilars or anti-IL-12/23 or any nonbiologic treatment with immunomodulatory properties such as JAK inhibitors within 90 days or 5 half-lives before baseline (whichever is longer).

### Permitted Concomitant Medications and Procedures

Medications and supplements including but not limited to vitamin E, betaine, s-adenosyl-1-methionine, ursodeoxycholic acid, statins, milk thistle, fibrates (e.g., gemfibrozil), probiotics, biguanides (metformin), bile acid sequestrants (e.g., cholestyramine or colestipol), TZDs, sodium-glucose co-transporter-2 (SGLT2) inhibitors, and GLP-1 RA that have been used to treat NAFLD/NASH are allowed during the course of the study if the participant has been on a stable dosing regimen (i.e., same dose and frequency in the previous 3 months before the qualifying liver biopsy and not initiated after qualifying liver biopsy) and is anticipated to maintain this dosing regimen throughout study participation. Use of antiplatelet medications is also allowed. The investigator must contact the contract research organization (CRO) medical monitor to discuss any changes to concomitant medications that may impact the study.

Participaants taking 5-aminosalicyclic acid, glucocorticoids, or immunosuppressants (e.g., azathioprine, 6-mercaptopurine, or methotrexate) for IMIDs should be on a stable dose for at least 12 weeks before screening and anticipated to remain stable throughout the study.

### Example 4: Study Intervention and Concomitant Therapy

### Study Intervention

The study drug is the MAdCAM-1 antibody (i.e., a fully human immunoglobulin G2 kappa antihuman MAdCAM-1 monoclonal antibody) which is provided as a sterile aqueous buffered solution for subcutaneously (SC) administration in a glass prefilled syringe (PFS) with a fixed needle. Each PFS contains 1 mL of the MAdCAM antibody solution for injection at a concentration of 75 mg/mL. The MAdCAM antibody solution is administered at Day 1 and Q4W thereafter for up to 24 weeks (last dose at Week 20). The MAdCAM-1 antibody is stored and maintained at temperature range of 2° C. to 8° C.

### Study Intervention Administration

The study intervention (i.e., the MAdCAM-1 antibody) is administered SC Q4W at the time points specified in the schedule of activities that includes treatment period, follow-up period and end of study (Table 8).

The study drug is administered in the anterolateral right or left thigh. The injection site is rotated. If there are clinical reasons why the drug cannot be administered in the thigh, the drug is administered in the deltoid area or abdomen with appropriate documentation. After the first administration of study drug, the participant is observed for at least 30 minutes.

### Example 5: Efficacy Assessments

Time points for all efficacy assessments are provided under schedule of activities in Table 8.

### Liver Stiffness Measurement and Liver Fat Content

Liver stiffness, as measured by elastography, is a marker for hepatic fibrosis. It is quantified by ultrasound-based transient elastography (FibroScan). Transient elastography correlates well with histology (Metavir and Ishak stages), performing best at extreme stages, i.e., severe fibrosis versus nonfibrosis. In addition, the rate of change over time has a correlation with clinical outcomes.

Assessments by FibroScan (LSM by FibroScan and liver fat content by CAP) is performed at the time points specified in Table 8. The results are read locally; the baseline reading at the first screening visit is used to calculate FAST scores to determine liver biopsy eligibility.

### FibroScan-Aspartate Aminotransferase Score

The FAST score has been developed to identify patients with NASH, elevated NAS score (NAS≥4), and advanced fibrosis (F≥2). The FAST score consists of liver stiffness measurement (LSM) by vibration-controlled transient elastography and controlled attenuation parameter (CAP), both measured by FibroScan, and AST. The FAST score provides an efficient way to non-invasively identify patients at risk of progressive NASH for clinical trials thereby reducing unnecessary liver biopsies.

A FAST score≥0.35, along with Pro-C3 levels>12.6 ng/ml and ELF score≥7.7 at the first screening visit (Week −8) trigger determination of eligibility for liver biopsy during screening. The FAST score is calculated by the following formula:

$$FAST = \frac{{e}^{- 1.65 + 1.07 \times ln ( LSM ) + 2.66 \times {10}^{- 8} \times {CAP}^{3} - 63.3 \times {AST}^{- 1}}}{1 + {e}^{- 1.65 + 1.07 \times ln ( LSM ) + 2.66 \times {10}^{- 8} \times {CAP}^{3} - 63.3 \times {AST}^{- 1}}}$$

To calculate the FAST score, LSM and CAP should come from a single FibroScan exam. The FibroScan exam and blood collection for AST assessment should be performed within 6 months.

The FAST score is calculated using the myFibroScan software. The FAST score is calculated at the at the time points specified in Table 8.

### Agile 4 Score

The Agile 4 score has been developed as a noninvasive test to identify cirrhosis in patients with NAFLD. The Agile 4 score is a FibroScan-derived score that includes LSM, AST, ALT, platelets, diabetes status, and gender. An Agile 4 score≥0.57 during screening is consistent with a high likelihood of F4 fibrosis (i.e., cirrhosis). Agile 4 was externally and independently validated to identify patients with cirrhosis with a positive predictive value that is superior to FIB-4 or LSM alone. Agile 4's rule-out and rule-in cut-offs are associated with a much smaller indeterminate zone compared to FIB-4 and LSM alone to rule out or rule in cirrhosis. Agile 4 could be used in clinical practice to identify patients in need of hepatocellular carcinoma and esophageal varices screening.

The Agile 4 score is calculated at the time points specified in Table 8.

### Fibrosis-4 Index

The FIB-4 index is a noninvasive test for liver fibrosis. The FIB-4 score consists of age, ALT, AST, and platelet count, and is calculated using the following formula: age [years]×AST [U/L]/(platelet count [10⁹/L]×√ALT [U/L]).

FIB-4 has utility in identifying participants with advances liver fibrosis and prognosticating mortality and liver-related outcomes. A FIB-4 score≥3.48, along with LSM≥20 kPa, at screening would indicate a high likelihood of F4 fibrosis (i.e., cirrhosis). The FIB-4 score is calculated at the at the time points specified in Table 8.

### Child-Pugh Score

The Child-Pugh score is a scoring system to measure the severity of chronic liver disease inclusive of cirrhosis. It consists of five clinical features, three of which assess the synthetic function of the liver (TBL level, serum albumin, and INR) and two of which are based on clinical assessment (degree of ascites and degree of hepatic encephalopathy). The Child-Pugh score is calculated at the time points specified in Table 8.

### Model for End-Stage Liver Disease

The original model for end-stage liver disease (MELD) score is a prospectively developed and validated chronic liver disease severity scoring system that uses a patient's laboratory values for serum bilirubin, serum creatinine, and the INR for prothrombin time to predict 3-month survival. In patients with cirrhosis, an increasing MELD score is associated with increasing severity of hepatic dysfunction and increased 3-month mortality risk.

The MELD score changes over time depending on the course of the chronic liver disease and ranges from 6 to 40; increasing scores indicate progression/worsening of disease. The MELD score will be calculated at the time points specified in Table 8.

### Liver Biopsy

Reasonable attempts should be made to acquire a needle core liver biopsy specimen using a 16 gauge needle of at least 2.0 cm (expected to have ≥11 portal tracts) in length. Specimens less than 1.5 cm will not be accepted for evaluation. If the first sample is not a minimum of 1.5 cm, a second core sample should be collected. The anatomical location (right or left liver lobe) and the specimen adequacy (needle size, tissue length) should be recorded. All of the collected biopsy tissues should be submitted for central pathology review.

Liver biopsy samples will be processed into formalin fixed paraffin embedded blocks, routinely stained with hematoxylin and eosin (H&E) and Masson's trichrome (MTR) by a central laboratory. Stained slides will be used for confirmation of the diagnosis of NASH, assessment and grading of NAS activity including scoring of steatosis, lobular inflammation, ballooning, as well as fibrosis using the NASH CRN scoring system by a hepatopathologist. The NASH CRN score and its components (i.e., lobular inflammation, hepatocyte ballooning, steatosis grade) and NAS will be used for comparison of pre-versus post treatment.

A previous biopsy conducted ≤6 months and ≥1 month before the start of the screening period (or ≤3 months before the start of the screening period for any F4 participants who met the inclusion and exclusion criteria before establishment of safety and tolerability in the first four F2/F3 participants) as part of standard of care that meets the above criteria for an adequate liver biopsy may substitute for a fresh biopsy, provided sufficient numbers of sections (stained or unstained) and uncut biopsy block, as outlined in the histopathology manual are made available to the sponsor. If the previous biopsy is found to be inadequate by the study pathologist, a fresh biopsy will be required. The Week 24 liver biopsy should be taken from the same lobe as the screening biopsy.

Leftover tissue samples may be retained and stored for RNA sequencing (transcriptomics) analysis or other possible additional future analyses. Any retained samples will be de-identified and will not be labeled with patient identifying information. After 15 years from the end of the study, or earlier as required by local regulations, samples may be returned to the sponsor repository or destroyed per local regulations. Additionally, with the participant's consent, samples may be used for further research by the sponsor or others, such as universities or other companies, to contribute to the understanding of NASH or other diseases, the development of related or new treatments, or development of research methods.

### Multiparametric Magnetic Resonance Imaging: Iron-Corrected T1

LiverMultiScan® MRI is a fast, contrast-free MRI scan that provides quantitative assessment of the whole liver tissue by measuring biomarkers for fibrosis and inflammation, fat and iron. As part of the LiverMultiScan® technology, the owner company segments an entire slice of the liver parenchyma and quantifies iron-corrected T1 (cT1) relaxation timevalues across that slice of liver tissue, so capturing disease heterogeneity in analysis. The cT1 measurement has shown to be correlated with histopathological hallmarks of NASH. The abdominal MRI (to obtain liver cT1 isperformed at the time points specified in Table 8.

### HepQuant Shunt Disease Severity Index

The dual cholate test (HepQuant®-SHUNT) is an investivational assay. The test yields a DS) that quantifies global liver function and physiology through the measurement of hepatic filtration rates that are defined from clearances of cholic acid-24-¹³C (20 mg administered intravenously; “systemic”) and cholic acid-2,2,4,4-d4 (40 mg administered orally; “portal”). The HepQuant-SHUNT DSI quantifies hepatic impairment and is reproducible over a broad spectrum of etiologies of liver disease, stages of fibrosis, and clinical severity. The HepQuant SHUNT DSI is optional if the site is unable to perform this assessment but is not optional for the participant if he site has the capability to perform this assessment. The HepQuant-SHUNT DSI is performed at the time points specified in Table 8.

### Alcohol Screening

The Alcohol Use Disorders Identification Test (AUDIT) is a 10-item screening tool developed by the World Health Organization (WHO) to assess alcohol consumption, drinking behaviors, and alcohol-related problems (nida.nih.gov/sites/default/files/audit.pdf). Both a clinician-administered version and a self-report version of the AUDIT are included in this assessment. Participants will be encouraged to answer the AUDIT questions in terms of standard drinks. A score of 8 or more is considered to indicate hazardous or harmful alcohol use. The AUDIT has been validated across genders and in a wide range of racial/ethnic groups and is well suited for use in primary care settings.

The AUDIT will be performed at the time points specified in Table 8.

### Additional Biomarkers

C-reactive protein and calprotectin are key biomarkers of inflammation. There is a significant relationship between hsCRP and risk of disease progression in NAFLD and NASH. Yoneda et al., “High-sensitivity C-reactive protein is an independent clinical feature of nonalcoholic steatohepatitis (NASH) and also of the severity of fibrosis in NASH”, J Gastroenterol, 42(7), 573-82 (2007). Pro C3 is a key biomarker of liver fibrosis. Mixed acting anti-metabolic agents (FGF19/21 analogues) that affect inflammatory pathways (e.g., NF-KB) decreased Pro-C3 within short trials ≤16 weeks by at least 20%, which is considered a clinically significant change (i.e., associated with improvement in liver fibrosis by ≥1 stage improvement in longitudinal studies). Luo et al., “An evaluation of the collagen fragments related to fibrogenesis and fibrolysis in nonalcoholic steatohepatitis”, Sci Rep, 8(1), 12414 (2018). In the open label, 12-week Phase 2a trial with aldafermin, levels of Pro-C3 and ELF had greater reductions in patients who demonstrated histological response (histological response was defined as a 2-point or greater improvement in NAS without worsening of fibrosis or improvement in fibrosis of one stage or more without worsening of NASH [defined as no increase in NAS for ballooning, inflammation, or steatosis]), compared to non-responders. Harrison et al., “Efficacy and safety of aldafermin, an engineered FGF19 analog, in a randomized, double-blind, placebo-controlled trial of patients with nonalcoholic steatohepatitis”, Gastroenterology, 160(1), 219-31.el (2021). Biomarkers of liver disease such as ELF will be derived as a composite score from serum levels of TIMP-1, PIIINP, and HA. The ELF is the first prognostic tool for patients with advanced fibrosis (F3 or F4) due to NASH to be granted de novo marketing authorization by FDA. Elevated ELF levels are prognostic for progression to cirrhosis (ELF≥9.8) and liver-related clinical events (ELF≥11.3) in NASH patients with advanced fibrosis. Fagan et al., “ELF score≥9.8 indicates advanced hepatic fibrosis and is influenced by age, steatosis and histological activity”, Liver Int, 35(6), 1673-81 (2015); Harrison et al., “Prospective validation of the enhanced liver fibrosis (ELF) test for the prediction of disease progression in patients with nonalcoholic steatohepatitis (NASH) and advanced fibrosis”, Abstract No. 2122. Hepatology, 66, 1120A-1A (2017) Magnetic resonance imaging-proton density fat fraction (MRI-PDFF) is a part of Liver MultiScan® and has an excellent correlation with histological steatosis across the spectrum of NAFLD and high diagnostic accuracy in stratifying all grades of hepatic steatosis (Dennis et al. 2021). Spleen cT1 significantly correlates with the hepatic venous pressure gradient (HVPG) and has an excellent diagnostic accuracy for portal hypertension (HVPG>5 mmHg) and clinically significant portal hypertension (HVPG≥10 mmHg) with an area under the receiver operating characteristic curve of 0.92 for both. Levick et al., “Non-invasive assessment of portal hypertension by multi-parametric magnetic resonance imaging of the spleen: A proof of concept study”, PLOS One, 14(8), e0221066 (2019).

Blood and liver biopsy samples are collected at the time points specified in the Table 8.
Serum Pro-C3
Serum ELF and its components (TIMP-1, PIIINP, and HA)
Serum soluble MAdCAM-1
Serum and plasma markers of inflammation, fibrosis, and trafficking including but not limited to: hsCRP, calprotectin (plasma), IL-8, CTXIII, C3M, TSP2, and Pro-C5
Exploratory analyses in liver biopsies including but not limited to markers of inflammation, fibrosis, and artificial intelligence/machine learning methods
T and immune cell trafficking in liver biopsy samples (immunohistochemistry) and circulating whole blood samples, including but not limited to Th17/Th1 vs. Th2, β₇⁺ T cells, CCR9, and CXCR3
Transcriptomics using residual liver biopsies and whole blood samples

Blood samples must be collected before administration of study drug. The ELF score is calculated from the concentrations of serum biomarkers: TIMP-1, PIIINP, and HA.

The samples may be stored for up to 15 years after the end of the study to achieve study objectives. Additionally, with the participant's consent, samples may be used for further research by the sponsor or others, such as universities or other companies, to contribute to the understanding of NASH or other diseases, the development of related or new treatments, or development of research methods.

### Example 6: Safety Assessments

Time points for all safety assessments are provided under schedule of activities in Table 8.

### Targeted Neurological Assessment

Targeted neurological assessments to monitor the development of signs and/or symptoms of progressive multifocal leukoencephalopathy (PML) is performed at the time points specified in Table 8. Participants are evaluated to reveal any potential abnormalities in the following neurological domains: vision, motor, tactile sensation, coordination/cerebellar function, speech, verbal comprehension, and cognition/behavior.

If any abnormalities are indicated, participants will be further evaluated to help clarify any potential abnormal responses. Focus will be placed on possible alternative etiology (eg, fracture or stroke). If additional evaluation reveals an unexplained new abnormality, neurological examination(s), targeted to the abnormal domain, will be performed by the investigator or qualified personnel.

### Hypersensitivity Monitoring

Participants are monitored for the presence of Type I (anaphylaxis) and Type III (immune complex) hypersensitivity reactions (e.g., fever, rash including hives, arthralgia, myalgia, vasculitis, Arthus reaction, general ill feeling, itching, and swollen lymph nodes) at the time points specified in Table 8.

Participants who experience an adverse event (AE) suggestive of a Type I hypersensitivity reaction have a blood sample (3 mL) collected for anti-MAdCAM-1 immunoglobulin E antibody determination. Participants who have a suspected Type III hypersensitivity reaction have a blood sample (4 mL) collected for complement determination (C3a, C5b-9).

### Pharmacokinetics

A blood sample for determination of serum MAdCAM-1 antibody concentrations is collected at the time points specified in Table 8. Blood samples is collected before study intervention administration. Details of sample collection, handling, shipment, and bioanalysis is provided in the laboratory manual.

### Immunogenicity Assessments

A blood sample for measurement of antidrug antibody (ADA) and neutralizing antibodies (NAb) is collected at the time points specified in Table 8. Blood samples are collected before administration of the study intervention at that visit. The detection and characterization of ADA and NAb are performed using a validated assay method

### Example 7: Endpoints Related To Each Objective

Respective endpoints of the study are listed against each objective in Table 10.

TABLE 10
Endpoints for Each Objectives

| | |
|---|---|
| Objectives | Endpoints |

Primary

| | |
|---|---|
| To evaluate safety and tolerability of | Incidence of treatment-emergent |
| aMAdAMD-1 antibody 75 mg administered | adverse events (TEAEs) |
| Q4W in participants with NASH (defined | Number of participants with clinically |
| as NAS ≥ 4 with at least 1 point each in | significant changes in the following |
| steatosis, ballooning, and lobular | parameters from baseline to the end of |
| inflammation) with different fibrosis stages | the follow-up period (Week 36): |
| (NASH Clinical Research Network [CRN] | Laboratory tests |
| F2, F3, and F4cc participants). | Electrocardiograms (ECGs) |
| | Vital signs |
| | Body weight |

Secondary

| | |
|---|---|
| To determine if the changes from baseline | Percent change from baseline in Pro- |
| in key biomarkers (neoepitope-specific N- | C3 through Week 24 |
| terminal propeptide of type III collagen | Percent change from baseline in ELF |
| [Pro-C3] and Enhanced Liver Fibrosis test | through Week 24 |
| [ELF]) and in cT1 MRI (iron-corrected T1 | Change from baseline in liver cT1 |
| mapping by magnetic resonance imaging) | MRI at Week 24 |
| provide a signal of a potential role of the | |
| MAdCAM-1 pathway in participants with | |
| NASH (defined as NAS ≥ 4 with at least | |
| 1 point each in steatosis, ballooning, and | |
| lobular inflammation) with different | |
| fibrosis stages (NASH CRN F2, F3, and | |
| F4cc participants) after 24 weeks of | |
| administration of MAdCAM-1 antibody | |
| 75 mg Q4W. | |

Exploratory

| | |
|---|---|
| To evaluate the changes from baseline in | Change from baseline to Week 24 for each |
| soluble MAdCAM-1 after 24 weeks of | fibrosis stage group (fibrosis stage 2, 3, and |
| administration of MAdCAM-1 antibody 75 | 4) and overall in soluble MAdCAM-1. |
| mg Q4W in participants with NASH (NAS ≥ | |
| 4 with at least 1 point each in steatosis, | |
| ballooning, and lobular inflammation) in | |
| each of the different fibrosis stages (NASH | |
| CRN F2, F3, and F4cc participants) and | |
| overall. | |
| To evaluate the changes from baseline in | Change from baseline to Week 24 for each |
| inflammatory and cell trafficking markers | fibrosis stage group (fibrosis stage 2, 3, and |
| in liver biopsy and blood samples after 24 | 4) and overall in: |
| weeks of administration of MAdCAM-1 | Serum or plasma biomarkers including |
| antibody 75 mg Q4W in participants with | but not limited to: high-sensitivity C- |
| NASH (NAS ≥ 4 with at least 1 point each | reactive protein (hsCRP) (serum), |
| in steatosis, ballooning, and lobular | interleukin (IL)-8 (serum), and |
| inflammation) in each of the different | calprotectin (plasma) |
| fibrosis stages (NASH CRN F2, F3, and | T and immune cell trafficking in liver |
| F4cc participants) and overall. | biopsy samples and circulating whole |
| | blood samples, including but not |
| | limited to: Th17/Th1 vs. Th2, β₇⁺ |
| | T cells, CCR9, and C-X-C motif |
| | chemokine receptor 3 (CXCR3) |
| | Liver biopsy exploratory analyses |
| | including but not limited to markers of |
| | inflammation, fibrosis and artificial |
| | intelligence/machine learning methods |
| | Transcriptomics in liver biopsy and |
| | whole blood samples |
| To evaluate the changes in fibrosis markers | Change from baseline to Week 24 for each |
| from baseline after 24 weeks of | fibrosis stage group (fibrosis stage 2, 3, and |
| administration of MAdCAM-1 antibody 75 | 4) and overall in: |
| mg Q4W in participants with NASH (NAS ≥ | Liver stiffness measure (LSM) by |
| 4 with at least 1 point each in steatosis, | FibroScan |
| ballooning, and lobular inflammation) in | Fibrosis-4 Index (FIB-4) |
| each of the different fibrosis stages (NASH | FibroScan-aspartate aminotransferase |
| CRN F2, F3, and F4cc participants) and | (FAST) score |
| overall. | |
| To evaluate the changes in liver fat content | Change from baseline to Week 24 for each |
| from baseline after 24 weeks of | fibrosis stage group (fibrosis stage 2, 3, and |
| administration of MAdCAM-1 antibody 75 | 4) and overall in proton density fat fraction |
| mg Q4W in participants with NASH (NAS ≥ | (PDFF) as measured by MRI-PDFF, part of |
| 4 with at least 1 point each in steatosis, | the Liver MultiScan ®. |
| ballooning, and lobular inflammation) in | |
| each of the different fibrosis stages (NASH | |
| CRN F2, F3, and F4cc participants) and | |
| overall. | |
| To evaluate the changes in spleen cT1 MRI | Change from baseline to Week 24 for each |
| from baseline after 24 weeks of | fibrosis stage group (fibrosis stage 2, 3, and |
| administration of MAdCAM-1 antibody 75 | 4) and overall in spleen cT1 MRI. |
| mg Q4W in participants with NASH (NAS ≥ | |
| 4 with at least 1 point each in steatosis, | |
| ballooning, and lobular inflammation) in | |
| each of the different fibrosis stages (NASH | |
| CRN F2, F3, and F4cc participants) and | |
| overall. | |
| To evaluate the changes from baseline in | Change from baseline to Week 24 for each |
| liver histology concerning NASH CRN | fibrosis stage group (fibrosis stage 2, 3, and |
| fibrosis stage (lobular inflammation grade, | 4) and overall in: |
| hepatocyte ballooning grade, steatosis | Lobular inflammation grade |
| grade, NAS) after 24 weeks of | Hepatocyte ballooning grade |
| administration of MAdCAM-1 antibody 75 | Steatosis grade |
| mg Q4W in participants with NASH (NAS ≥ | NAS |
| 4 with at least 1 point each in steatosis, | |
| ballooning, and lobular inflammation) in | |
| each of the different fibrosis stages (NASH | |
| CRN F2, F3, and F4cc participants) and | |
| overall. | |
| To evaluate the changes from baseline in | Change from baseline to Week 24 for each |
| liver function and biochemistry after 24 | fibrosis stage group (fibrosis stage 2, 3, and |
| weeks of administration of MAdCAM-1 | 4) and overall in: |
| antibody 75 mg Q4W in participants with | Alanine aminotransferase (ALT) |
| NASH (NAS ≥ 4 with at least 1 point each | Aspartate aminotransferase (AST) |
| in steatosis, ballooning, and lobular | AST/ALT ratio |
| inflammation) in each of the different | Alkaline phosphatase (ALP) |
| fibrosis stages (NASH CRN F2, F3, and | Gamma-glutamyl transferase |
| F4cc participants) and overall. | Total bilirubin (TBL) |
| | International normalized ratio (INR) |
| | Albumin |
| | HepQuant-SHUNT-Disease Severity |
| | Index (DSI) |
| To evaluate the pharmacokinetics (PK) of | Circulating serum concentrations of |
| MAdCAM-1 antibody 75 mg Q4W in | MAdCAM-1 over the treatment period. |
| participants with NASH (NAS ≥ 4 with at | |
| least 1 point each in steatosis, ballooning, | |
| and lobular inflammation) (NASH CRN F2, | |
| F3, and F4cc participants). | |
| To evaluate immunogenicity of MAdCAM- | Incidence of formation of antidrug |
| 1 antibody 75 mg Q4W in participants with | antibodies (ADAs) through the end of the |
| NASH (NAS ≥ 4 with at least 1 point each | follow-up period (Week 36). |
| in steatosis, ballooning, and lobular | |
| inflammation) (NASH CRN F2, F3, and | |
| F4cc participants). | |

### Example 8: Second Study Design and Methodology

The second study is a multi-dose placebo controlled study. Briefly, the study includes subjects having the CRN fibrosis score 2 or 3 and NAS≥4 with at least 1 point each in inflammation and ballooning.

The study includes dose ranging 25 mg, 75 mg, or 150 mg of MAdCAM-1 antibody, or placebo, randomized 1:1:1:1 or 2:1:2:2 ratio. The treatment duration ranges 52-72 weeks.

The primary endpoint of the study requires improvement in fibrosis (≥1 stage) with no worsening of NASH; and/or resolution of NASH (NAS score of 0-1 for inflammation, 0 for ballooning, and any value for steatosis) without worsening fibrosis.

Some important secondary endpoints include inflammatory markers, liver biochemistry, MRI-cT1, Fibroscan, PK, PROs, HRQOL, and health care utilization.

### Example 9: Third Study Design and Methodology

The third study is a histology study that includes subjects having the CRN fibrosis score 2 or 3 and NAS≥4 with at least 1 point each in inflammation and ballooning.

The study includes the selected dose from the second study against placebo or the standard of care. The treatment duration lasts up to 104 weeks.

The primary endpoint of the study requires improvement in fibrosis (≥1 stage) with no worsening of NASH; and/or resolution of NASH (NAS score of 0-1 for inflammation, 0 for ballooning, and any value for steatosis) without worsening fibrosis.

Some important secondary endpoints include improvement in fibrosis (≥1 stage) with no worsening of NASH. Endpoint also includes inflammatory markers, liver biochemistry, MRI-cT1, Fibroscan, PK, PROs, HRQOL, and health care utilization.

### Example 10: Fourth Biomarker Study Design and Methodology

The fourth study is a biomarker study that includes NASH subjects based on non-invasive markers and elevated biomarkers.

The study includes the selected dose from the second study. The treatment duration lasts for 52 weeks.

The primary endpoint of the study requires fibrosis and inflammatory markers.

Some important secondary endpoints include liver biochemistry and metabolic parameter.

### Example 11: Fifth Study Design and Methodology

The fifth study is a post-approval outcome study that includes subjects having the CRN fibrosis score 2 or 3 and NAS≥4 with at least 1 point each in inflammation and ballooning. Patients roll over from Phase 3 histology study.

The study includes the selected dose from the second study against placebo or the standard of care. The treatment duration ranges 3-5 years.

The primary endpoint of the study includes progression to cirrhosis on histopathology, reduction in hepatic decompensation events, change in MELD score from ≤12 to >15, liver transplant, and all-cause mortality.

Some important secondary endpoints include inflammatory markers, liver biochemistry, MRI-cT1, Fibroscan, PK, PROs, HRQOL, and health care utilization.

### Example 12: Sixth Study Design and Methodology

The sixth study is a long-term safety study which includes subjects who roll over from the second, third, fourth, and/or fifth study. The study includes the selected dose from the second study. The treatment duration lasts until the marketing approval.

The primary endpoint of the study includes safety (AE, SAE, etc.) and product specific safety endpoints.

Some important secondary endpoints include inflammatory markers, inflammatory markers, liver biochemistry, MRI-cT1, and health care utilization.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method for the treatment of a patient susceptible to or diagnosed with non-alcoholic steatohepatitis comprising administering to the patient a therapeutic dose of between 20 mg and 125 mg of a MAdCAM-1 antibody.

2. The method of claim 1, further comprising administering a subsequent dose of a MAdCAM-1 antibody.

3. (canceled)

4. The method of claim 1, comprising administering to the patient the therapeutic dose of 22.5 mg or 75 mg of MAdCAM-1 antibody.

5. (canceled)

6. The method of claim 1, wherein the MAdCAM-1 antibody comprises a light chain CDR1 of SEQ ID NO: 11; a light chain CDR2 of SEQ ID NO: 12; and a light chain CDR3 of SEQ ID NO: 13; and a heavy chain CDR1 of SEQ ID NO: 14; a heavy chain CDR2 of SEQ ID NO: 15; and a heavy chain CDR3 of SEQ ID NO: 16.

7. The method of claim 6, wherein the MAdCAM-1 antibody comprises a variable light chain of SEQ ID NO: 3, and a variable heavy chain of SEQ ID NO: 4.

8. (canceled)

9. The method of claim 2, wherein the subsequent dose is administered at an amount that is same or less than the therapeutic dose, and the subsequent dose is administered between about 1 week and about 12 weeks after the therapeutic dose.

10.-11. (canceled)

12. The method of claim 1, wherein the MAdCAM-1 antibody is administered to the patient subcutaneously or intravenously.

13. (canceled)

14. The method of claim 1, wherein the patient is not taking a TNF antagonist or TNF inhibitor.

15. A MAdCAM-1 antibody for use in a method of claim 1.

16. A pharmaceutical composition comprising the MAdCAM-1 antibody of claim 15.

17. (canceled)

18. A method for the treatment of a patient susceptible to or diagnosed with non-alcoholic steatohepatitis comprising administering to the patient a therapeutic dose of 22.5 mg or 75 mg of a MAdCAM-1 antibody comprising a light chain CDR1 of SEQ ID NO: 11; a light chain CDR2 of SEQ ID NO: 12; and a light chain CDR3 of SEQ ID NO: 13; and a heavy chain CDR1 of SEQ ID NO: 14; a heavy chain CDR2 of SEQ ID NO: 15; and a heavy chain CDR3 of SEQ ID NO: 16.

19. (canceled)

20. The method of claim 19, wherein the MAdCAM-1 antibody comprises a variable light chain of SEQ ID NO: 3, and a variable heavy chain of SEQ ID NO: 4.

21. (canceled)

22. The method of claim 18, further comprising administering a subsequent dose, wherein the subsequent dose is administered at an amount that is same or less than the therapeutic dose, and the subsequent dose is administered between about 1 week and about 12 weeks after the therapeutic dose.

23. (canceled)

24. The method of claim 18, wherein the MAdCAM-1 antibody is administered to the patient subcutaneously or intravenously.

25. A method for assessing the presence or absence of a beneficial response in a patient with non-alcoholic steatohepatitis with different fibrosis stages after administering subcutaneously to the patient a dose of 22.5 mg or 75 mg of the MAdCAM-1 antibody of claim 15, comprising:
(a) measuring the level of a biomarker in a biological sample from said patient, wherein said biomarker is: (i) neoepitope-specific N-terminal pro-peptide of type III collagen (Pro-C3); and (ii) Enhanced Liver Fibrosis (ELF); and
(b) comparing said level with a control;
wherein a change in the level of biomarker, as compared to the control, is predictive of a beneficial response in said patient.

26. A method for the treatment of a patient susceptible to or diagnosed with non-alcoholic steatohepatitis comprising administering to the patient a therapeutic dose of between 25 mg and 150 mg of a MAdCAM-1 antibody.

27. The method of claim 26, further comprising administering a subsequent dose of a MAdCAM-1 antibody.

28. (canceled)

29. The method of claim 26, comprising administering to the patient the therapeutic dose of 25 mg, 75 mg, or 150 mg of MAdCAM-1 antibody.

30.-36. (canceled)

37. The method of claims 27, wherein the subsequent dose is administered between about 1 week and about 104 weeks after the therapeutic dose.

38. A method for the treatment of a patient susceptible to or diagnosed with non-alcoholic steatohepatitis comprising administering to the patient a therapeutic dose of 25 mg, 75 mg, or 150 mg of a MAdCAM-1 antibody comprising a light chain CDR1 of SEQ ID NO: 11; a light chain CDR2 of SEQ ID NO: 12; and a light chain CDR3 of SEQ ID NO: 13; and a heavy chain CDR1 of SEQ ID NO: 14; a heavy chain CDR2 of SEQ ID NO: 15; and a heavy chain CDR3 of SEQ ID NO: 16.

39.-41. (canceled)


SEQUENCE LISTING (38 sequences)

SEQ ID NO: 1 - protein, 219 aa, synthetic construct
  REGION 1..219: note = Synthetic Construct
DIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, synthetic construct
  REGION 1..450: note = Synthetic Construct
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 112 aa, synthetic construct
  REGION 1..112: note = Synthetic Construct
DIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIK

SEQ ID NO: 4 - protein, 124 aa, synthetic construct
  REGION 1..124: note = Synthetic Construct
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSS

SEQ ID NO: 5 - protein, 107 aa, synthetic construct [sequence omitted]

SEQ ID NO: 6 - protein, 101 aa, synthetic construct
  REGION 1..101: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERK

SEQ ID NO: 7 - protein, 9 aa, synthetic construct
  REGION 1..9: note = Synthetic Construct
CCVECPPCP

SEQ ID NO: 8 - protein, 109 aa, synthetic construct
  REGION 1..109: note = Synthetic Construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 9 - protein, 107 aa, synthetic construct
  REGION 1..107: note = Synthetic Construct
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 16 aa, synthetic construct
  REGION 1..16: note = Synthetic Construct
KSSQSLLHTDGTTYLY

SEQ ID NO: 12 - protein, 7 aa, synthetic construct
  REGION 1..7: note = Synthetic Construct
EVSNRFS

SEQ ID NO: 13 - protein, 9 aa, synthetic construct
  REGION 1..9: note = Synthetic Construct
MQNIQLPWT

SEQ ID NO: 14 - protein, 5 aa, synthetic construct
  REGION 1..5: note = Synthetic Construct
SYGIN

SEQ ID NO: 15 - protein, 17 aa, synthetic construct
  REGION 1..17: note = Synthetic Construct
WISVYSGNTNYAQKVQG

SEQ ID NO: 16 - protein, 15 aa, synthetic construct
  REGION 1..15: note = Synthetic Construct
EGSSSSGDYYYGMDV

SEQ ID NO: 17 - protein, 218 aa, synthetic construct
  REGION 1..218: note = Synthetic Construct
DIVMTQSPLSLPVTPGEPASISCRSSQSLLQSNGYNYLDWYLQKPGQSPQLLIYLGSNRASGVPDRFSGSGSGTDFTLKISRVEAEDVGVYYCMQALQTITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 444 aa, synthetic construct
  REGION 1..444: note = Synthetic Construct
EVQLVESGGGLVKPGGSLRLSCVASGFTFTNAWMIWVRQAPGKGLEWVGRIKRKTDGGTTDYAAPVKGRFTISRDDSKNTLYLQMNSLKTEDTAVYYCTTGGVAEDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 218 aa, synthetic construct
  REGION 1..218: note = Synthetic Construct
DIVMTQSPLSLPVTPGEPASISCRSSQSLLQSNGFNYLDWYLQKPGQSPQLLIYLGSNRASGVPDRFSGSGSGTDFTLKISRVEAEDVGVYYCMQALQTITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 444 aa, synthetic construct
  REGION 1..444: note = Synthetic Construct
EVQLVESGGGLVKPGGSLRLSCVVSGFTFTNAWMIWVRQAPGKGLEWVGRIKRKTDGGTTDYAAPVKGRFTISRDDSKNTLYLQMNSLKTEDTAVYYCTTGGVAEDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 214 aa, synthetic construct
  REGION 1..214: note = Synthetic Construct
DIQMTQSPSSLSASVGDRVTITCRASRSISSYLNWYQQKPGKAPKVLIFFVSSLQSGVPSRFSGSGSGTDFTLTISSLQPEDFATYYCQQNYIPPITFGQGTRLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 448 aa, synthetic construct
  REGION 1..448: note = Synthetic Construct
EVQLLESGGGLVQPGGSLRLSCAASGLTFNNSAMTWVRQAPGKGLEWVSTTSGSGGTTYYADSVKGRFTISRDSPKNTLYLQMNSLRAEDTAVYYCAARGYSYGTTPYEYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 214 aa, synthetic construct
  REGION 1..214: note = Synthetic Construct
EIVLTQSPDFQSVTPKEKVTITCRASQRIGSSLHWYQQKPDQSPKLLIKYASQSFSGVPSRFSGSGSGTNFTLTINGLEAEDAATYYCHQSGRLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 446 aa, synthetic construct
  REGION 1..446: note = Synthetic Construct
QVQLVESGGGVVQPGRSLRLSCAASGHTFSSDGMHWVRQAPGKGLEWVAIIWYDGSNKYYADSVKGRFTISRDNSKNTLYLQMNSLRAEDTAVYYCARDPGYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTAPPVLDSDGSFFLYSRLTVDKSRWQEGNVFSCSVMHEALHNHYTQKSLSLSLGK

SEQ ID NO: 25 - protein, 214 aa, synthetic construct
  REGION 1..214: note = Synthetic Construct
DIQMTQSPSSLSASVGDRVTITCRASQNISSYLNWFQQKPGKAPKLLIYAASGLKRGVPSRFSGSGSGTDFTLTIRTLQPDDFATYSCHQSYSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 450 aa, synthetic construct
  REGION 1..450: note = Synthetic Construct
QVQLVESGGGVVQPGRSLRLSCAASGFTFSSYGMHWVRQAPGKGLEWVAVISNDGNNKYYADSVKGRFTISRDNSKNTLYLQMNSLSAEDTAVYYCARDSTAITYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 221 aa, synthetic construct
  REGION 1..221: note = Synthetic Construct
DIVMTQSPDSLAVSLGERATINCKSSQSVLYSSNNKTYLAWYQQKPRQPPKLLIYWASIREYGVPDRFSGSGSGTDFTLTISSLQAEDVAVYFCQQYYSIPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 451 aa, synthetic construct
  REGION 1..451: note = Synthetic Construct
QVQLQESGPGLVKPSETLSLTCTVSGDSISSNYWSWIRQPAGKGLEWIGRIYTSGGTNSNPSLRGRVTILADTSKNQFSLKLSSVTAADTAVYYCARDRITIIRGLIPSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 216 aa, synthetic construct
  REGION 1..216: note = Synthetic Construct
DIQMTQSPSSLSASVGDRVTFTCRASQNITNYLNWYQQKPGKAPKLLIYAASSLPRGVPSRFRGSGSGTDFTLTISSLQPEDFATYYCQQSYSNPPECGFGQGTTLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 452 aa, synthetic construct
  REGION 1..452: note = Synthetic Construct
EVQLLESGGDLVQPGGSLRLSCAASGFTFRSYAMNWVRQAPGKGLEWVSVISGRGGTTYYADSVKGRFTISRDNSKNTLYLQMNSLRAEDAAVYYCAKIAVAGEGLYYYYGMDVWGQGTTVTVSSASTKGPSVFPLAPCSRSTSENTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 219 aa, synthetic construct
  REGION 1..219: note = Synthetic Construct
DIVMTQTPLSLSVTPGQPASISCNSSQSLLLSDGKTYLNWYLQKPGQPPQLLIYEVSNRFSGVPDRFSGSGSGTDFTLKISRVEAEDVGVYSCMQSIQLMCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 453 aa, synthetic construct
  REGION 1..453: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGYSSGWSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSRSVMHEALHNHYTQKSLSLSLGK

SEQ ID NO: 33 - protein, 218 aa, synthetic construct
  REGION 1..218: note = Synthetic Construct
DIVMTQSPLSLPVTPGEPASISCRSSQSLLHGNGYNYLDWYLQKPGQSPQLLIYLGSNRASGVPDRFSGSGSGTDFTLKISRVEAEDVGVYYCMQALQTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 452 aa, synthetic construct
  REGION 1..452: note = Synthetic Construct
QVQLQESGPGLVKPSETLSLTCTVSGSSISSYHWNWIRQPAGKGLEWIGRIYTSGSTNYNPSLKSRVTMSLDTSKNQFSLKLSSVTAADTAVYYCAREGVRYYYASGSYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 219 aa, synthetic construct
  REGION 1..219: note = Synthetic Construct
DIVMTQTPLSLSVTPGQPASISCKSNQSLLYSDGKTYLFWYLQKPGQPPQLLIYEVSNRFSGVPDRFSGSGSGTDFTLKISRVEAEDVGVYYCMQSIQLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 450 aa, synthetic construct
  REGION 1..450: note = Synthetic Construct
QVQLVQSGAEVKKPGASVKVSCEASGYTFTSYGIDWVRQAPGQGLEWMGWISVYSGNTNYAQKLQGRVTMSTDTSTSTAYMELRSLRSDDTAVYYCAREGSSSSGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 214 aa, synthetic construct
  REGION 1..214: note = Synthetic Construct
DIQMTQSPSSLSASVGDRVTITCQASQDISNYLNWYQQKPGKAPKLLIYDASNLETGVPSRFSGSGSGTDFTFTISSLQPEDIATYSCQHSDNLSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 443 aa, synthetic construct
  REGION 1..443: note = Synthetic Construct
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARGAYHFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK